(19) United States
(12) Patent Application Publication
(10) Pub. No.: US 2024/0159755 A1
(43) Pub. Date: May 16, 2024

(54) DETECTION OF STAGE I LUNG CANCER BIOMARKERS

(71) Applicant: THE JOHNS HOPKINS UNIVERSITY, Baltimore, MD (US)

(72) Inventors: Ciprian M. Crainiceanu, Baltimore, MD (US); Lonny B. Yarmus, Clarksville, MD (US); Ana Maria Rule, Baltimore, MD (US)

(21) Appl. No.: 18/548,767

(22) PCT Filed: Mar. 1, 2022
(86) PCT No.: PCT/US2022/018356

§ 371 Date: Sep. 1, 2023

Related U.S. Application Data
(60) Provisional application No. 63/154,997, filed on Mar. 1, 2021.

(51) Int. Cl. G01N 33/574; A61B 5/08; A61B 10/00

(52) U.S. Cl. CPC G01N 33/57423; A61B 5/082; A61B 10/00; A61B 2010/0087

(57) ABSTRACT

VOCs in exhaled breath can be used to diagnose stage 1 lung cancer (SL1C). Three potential biomarkers Acetoin, Dodecane, and p-Cymene have predictive power for SL1C. Acetoin and Dodecane are predictive with relation to their concentrations in the 1 L breath sample, and p-Cymene is predictive 5 with relation to being above or below the limit of detection. The diagnostic of the present invention is capable of detecting S1LC noninvasively and in some cases earlier than other methodologies. This diagnostic can then be paired with appropriate treatments to address S1LC before it grows or metastasizes.

### CROSS REFERENCE TO RELATED APPLICATIONS

This application claims the benefit of U.S. Provisional Patent Application No. 63/154,997 filed on Mar. 1, 2021, which is incorporated by reference, herein, in its entirety.

### FIELD OF THE INVENTION

The present invention relates generally to diagnostic testing. More particularly, the present invention relates to a diagnostic test for detecting stage I lung cancer biomarkers.

### BACKGROUND OF THE INVENTION

Lung cancer is one of the most commonly occurring types of cancer, and it accounts for almost 25% of all cancer deaths. Treatment and long-term outcomes are dependent on the stage and type of lung cancer, as well as on the patient's health. While it is possible to diagnose lung cancer with medical imaging, it would be helpful to find additional diagnostic methods to allow for earlier diagnosis and treatment.

Regular screening in patients at risk has previously shown a mortality benefit and a patient's best chance of survival remains early detection. The National Lung Cancer Screening Trial (NLST) demonstrated a 20% relative decrease in lung cancer mortality with low dose CT scans (LDCT) with a sensitivity of 93.8%, specificity of 73.4%, and negative predictive value of 99.9%. Due to the results of this trial, LDCT scan has become the gold standard for early lung cancer detection. Despite these efforts, CT screening has suffered from slow adoption in part due to its 27% false positive rate which has led to unnecessary procedures with associated morbidity and mortality. Only 15% of lung cancer patients are diagnosed at an early stage. If detected at stage 1, the five-year survival can exceed 90%, thus additional early identification tests are needed.

It would therefore be advantageous to provide a new method for diagnosis of lung cancer, while it is in its earliest stage.

### SUMMARY OF THE INVENTION

In accordance with an embodiment, the present invention provides a method of detecting stage one lung cancer in a subject including collecting a breath sample from the subject. The method also includes analyzing the breath sample to detect at least one of Acetoin, Dodecane, and p-Cymene. The method further includes initiating a follow-up plan for the subject, if the at least one of Acetoin, Dodecane, and p-Cymene are detected.

In accordance with an aspect of the present invention, the method includes collecting multiple breath samples from the subject. The method includes using a device for analysis of the VOCs in the breath. The method includes using the device more than once, in order to confirm results. Additionally, the method includes collecting the breath sample in a bag or other receptacle. The bag or other receptacle takes the form of a Tedlar® bag or other film bag. The method includes analyzing the breath samples within 24 hours of collection, and in some instances includes analyzing the breath samples within 2 hours of collection. The method includes using a gas chromatograph for analysis of the breath sample. The follow up plan further includes additional testing, treatment, preventative and/or lifestyle changes.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a graphical view of an enrollment graph for the study as a function of time.

FIG. 2 illustrates graphical views of scatterplots of log 10 (peak) for Bag 1 (x-axis) versus Bag 2 (y-axis). Dark grey: regression line; light grey: identity line; axis labels: displayed on the original scale.

FIG. 3 illustrates graphical views of boxplots of peak area (left panel) and concentrations (right panel) on log 10 scale for Bag 1 (dark grey) and Bag 2 (light grey).

FIG. 4 illustrates graphical views of scatterplots of log 10 (concentration) for Bag 1 (x-axis) versus Bag 2 (y-axis). Dark grey: regression line; light grey: the identity line; axis labels: displayed on the original scale.

FIG. 5 illustrates graphical views of boxplots of log 10 (peak) for quantifiable VOCs.

FIG. 6 illustrates graphical views of boxplots of log 10(peak) for quantifiable VOCs separated by cases (dark grey), housemate controls (light grey), and matched controls (grey).

FIG. 7 illustrates graphical views of boxplots of log 10(peak) for quantifiable VOCs separated by cases (dark grey), housemate and matched controls combined (light grey).

FIG. 8 illustrates an info graphical view of correlation among VOC log 10 (peaks).

FIG. 9 compares VOC concentrations for training data separated by each control type and the two bags (left panel corresponds to Bag 1 and right panel corresponds to Bag 2).

FIG. 10 illustrates graphical views of boxplots of log 10 (concentrations) for quantifiable VOCs with concentrations above the limit of detection for at least 20% of measurements.

FIG. 11 illustrates a graphical view of classification based on Acetoin concentration threshold using the test data.

FIG. 12 illustrates graphical views of boxplots of log 10(peak) for unquantifiable VOCs separated by cases (dark grey), housemate and matched controls combined (grey).

FIGS. 13A and 13B illustrate graphical views of the distributions of VOC concentrations for training (FIG. 13A) and test (FIG. 13B) data separated by cases and control types.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

### Abbreviations

AUC=area under the receiver operating characteristic curve
Case=a study participant with biopsy-confirmed Stage I lung cancer
GC-MS=gas chromatograph-mass spectrometer
Groups=Biopsy confirmed stage I lung cancer study participant (control) with corresponding matched control (always available) and housemate control (when available)
Housemate control=a study participant identified as an adult from the household of the case patient with biopsy confirmed Stage I lung cancer
LOD=limit of detection
Matched control=a study participant from the population of patients who does not have a lung cancer diagnosis and is matched on demographic and behavioral variables with a biopsy-confirmed Stage I lung cancer study participant
PHI=Personal Health Information
PSI=Pounds per Square Inch
RSI=Reverse Search Index (Reverse Match Factor)
S1LC=Stage I lung cancer
SI=Similarity Index (Match Factor)
TD=thermal desorption
Test data=58 cases with matched and housemate controls used for validation of VOC biomarkers and models
Training data=30 cases with matched and housemate controls used for VOC biomarker discovery and model exploration
UASC=unique anonymous subject code
VOC=Volatile Organic Compounds

An invention in accordance with the present invention includes using VOCs in exhaled breath to diagnose stage 1 lung cancer (SL1C). Three potential biomarkers Acetoin, Dodecane, and p-Cymene have predictive power for SL1C. Acetoin and Dodecane are predictive with relation to their concentrations in the 1 L breath sample, and p-Cymene is predictive with relation to being above or below the limit of detection. The diagnostic of the present invention is capable of detecting S1LC non-invasively and potentially earlier than other methodologies. This diagnostic can then be paired with appropriate treatments to address S1LC before it grows larger or metastasizes.

The present invention is directed to concentrations of volatile organic compounds (VOCs) in the breath of biopsy-confirmed S1LC patients (cases) and lung-cancer-free individuals (controls). The present invention is specifically focused on S1LC, which is a very specific, early phase of lung cancer; (2) it uses calibration curves for thirteen compounds identified as potential biomarkers to obtain their concentrations expressed in μg/L; and (3) it uses a case to two control sampling design and additional protocol features to reduce the potential for bias, confounding, and measurement error.

Volatile organic compounds (VOCs) are carbon containing compounds which may be produced by the body, or be environmental contaminants. These compounds can be measured in breath, and there has been extensive research into their ability to be used for lung cancer diagnostics. The use of breath analytics in lung cancer has been previously attempted, each study with limitations. One of the first studies was in 1971 and identified 250 different VOCs in human breath samples. As technology advanced, this study was repeated in 1999 and over 3,000 breath VOCs were identified. Multiple studies have since followed and a re-emergence of a search for a VOC signature started in the 2010s. Fu et al. studied the differences in VOCs between lung cancer patients and healthy controls, with the goal to identify two or more VOCs as a “fingerprint” to identify lung cancer. They were able to identify a signature of 4 VOCs and reported a sensitivity and specificity of 89.9% and 81.3%, respectively. A second study was released a year later, which included an analysis of three groups of participants: benign nodules, healthy controls and lung cancer patients. This study reported a VOC signature with a sensitivity and specificity of 88.5% and 86.5%, respectively²¹. Multiple other studies have been conducted, none of which identified a VOC signature. None of these studies provided a practical and reproducible VOC signature of cancer as: (1) the definitions of “signatures” are impractical as they only provide a list of compounds; (2) data that underlined the results are not publicly available; (3) code that was used for signatures is not available; (4) VOCs are not expressed in units of concentration (e.g., μg/L); and (5) not enough practical details are provided about how to re-construct the “signature” in a new study. Moreover, many of these studies have been limited by small sample sizes, heterogeneous experimental conditions, lack of calibration of VOCs to the concentration scale, irreproducible analytic pipelines, and/or lack of built-in validation component. Despite being extensive, this existing literature does not provide a practical solution to detecting early-stage lung cancer using breath VOCs.

The present invention identifies VOCs signatures and quantify their discrimination properties, investigates whether simpler signatures (containing fewer VOCs) with large discrimination power exist and quantifies and ranks their discrimination power; and (3) searches for new VOC signatures and quantify their discrimination properties.

In order to implement the present invention, breath samples are obtained from the subject. The breath sample is analyzed for the VOCs that are indicative of S1LC. In some embodiments, it may be preferable for the subject to supply more than one sample for analysis. In other embodiments, it is possible that a device for analysis of the VOCs in the breath can be used. In such instances, the device can be used more than once, in order to confirm results. If the subject is to provide a sample for analysis, the breath sample can be provided in a bag or other receptacle known to or conceivable to one of skill in the art, such as a Tedlar® bag or other film bag. When possible, to avoid the effects of time on the samples, breath samples are analyzed within 24 hours of their collection, though, typically, the analysis is conducted within two hours of breath sample collection. In some embodiments, a gas chromatograph is used for analysis. If VOCs indicative of SL1C are found, additional steps can be taken by the diagnosis and treatment team. For instance, the diagnosis and treatment team might preform additional diagnostic testing, treatment, preventative and/or lifestyle changes, or any other treatment step known to or conceivable to one of skill in the art.

### EXAMPLES

The following examples and data are included herein by way of illustration of the invention. These examples are not meant to be considered limiting, and the invention is considered to include any implementation known to or conceivable to one of skill in the art.

A study used as a basis for the present invention has a matched case to two controls design with continuous enrollment. The first 30 cases with matched and housemate control trios are used to conduct preliminary exploratory analyses, sample size projections, and exploratory analyses of new VOC signatures. Some trios may not contain one of the controls and the resulting data are referred to as groups instead of trios to reflect this reality. Each participant provided two samples of breath air into Tedlar® bags or other film bags, one after the other without breathing in-between. The resulting samples are referred to as the bag 1 and bag 2 samples, respectively. For each participant and each bag sample all VOCs detectable by the lab equipment were described using two methods: (1) log-area under the curve peaks; and (2) concentration for a subset of 13 VOCs for which calibration curves could be obtained. These 13 VOCs are referred to as quantifiable VOCs and the rest as unquantifiable VOCs. In other applications, calibration curves can be available for a larger or smaller set of VOCs; therefore, the definition of quantifiable VOCs is specific to this study; a list of quantifiable VOCs are included, herein. The quantifiable VOCs were chosen among the ones identified in the VOC breath analysis literature, though calibration substances were available only for some of the published compounds. The quantifiable VOC data is used for the first 30 groups as training set for conducting cancer prediction modeling. The remaining groups (a total of 58 groups) are used for validation of findings.

A case is defined as a person with biopsy-confirmed S1LC. A control is defined as a person without lung cancer. Cases were identified from a population of lung cancer patients. For each case the plan was to identify 2 controls: (1) the first control (type 1: matched control) participant was identified from the population of patients who do not have a lung cancer diagnosis; (2) the second control (type 2: housemate control) participant was identified as another adult person from the household of the case patient who does not have a lung-cancer diagnosis. Collecting information from a type 2 control was not always possible either because another household adult was not available or, if one was available, they did not consent to participate in the study. The type 1 controls were identified via a medical records system based on covariate matching. The type 2 controls were identified, when possible, during the preliminary patient visit. Because some cases do not have a matched housemate control, some matched groups contain two study participants (case and type 1 control matched on covariates) and some contain three participants (case, type 1 control matched on covariates, and type 2 housemate control). These are referred to as groups instead of trios, as the matched groups do not always contain three study participants.

Matching was conducted to reduce the potential for confounding. Two types of controls are used for each case. All participants were asked to abstain from smoking, vaping, or drinking for at least 30 minutes before conducting the test. The type 1 control was identified via the medical records system and was matched to the lung cancer case patient using the following variables:
a. Smoking status (never, former, light smoker, heavy smoker).
   attempted to find a match with the same/similar number of cigarette pack-years. If that was not possible, enrolled a patient who matched in terms of being a current or former smoker
b. Vaping (yes, no).
   non-vapors were matched. While current vapors were difficult to find (given all other matching variables), the search was widened to include current smokers as a match
c. Sex (female, male)
   always matched
d. History of family lung cancer (yes, no)
   always matched
e. Age (±5 years)
   always matched
f. Race (Caucasian, African-American, Asian-American, Hispanic/Latino, other)
   for East Asian or Middle Eastern patients the clinical team was often unable to find a match. When this was the case, the search was widened to include all of Asia
g. Alcohol use (never, light, moderate, heavy)
   fairly well matched

The type 2 control is identified, when possible, during the preliminary patient visit and is an adult person who lives in the same household with the lung cancer case patient. Some patients did not have another adult living in the same household or if they have, they did not consent to participate in the study; in these cases the type 2 control sample was not collected.

To avoid possible analytic batch effects, the sampling for cases and controls was conducted as close in time as possible. For the type 2 controls, the sampling was done during the same visit, whenever possible. To avoid potential effects of time between sample collection and analysis, the lab received and analyzed the breath samples within 24 hours of their collection, though, typically, the analysis was conducted within two hours of breath sample collection. In 11 samples the time between collection and analysis was between 24 and 32 hours, and in 3 samples the time between collection and analysis was between 6 and 11 days. The sampling and analysis of cases and controls was not conducted in separate groups to avoid temporal batch effects. The VOC analysis laboratory did not receive information about case status.

Every study participant was assigned a unique anonymous subject code (UASC). Subject identifying data (e.g., name) and the link to the UASC data were stored securely by the team. Subject-identifying data was not requested or shared with the laboratory team. Quality control was assured by direct collaboration between the teams. Each of the three teams identified a team member who was in charge of data quality control. All data were recorded in a database system, compliant with all Personal Health Information (PHI) regulations. Quality control was conducted in a multilayer approach and in close collaboration between the team members to: (1) correct typos and incorrect coding; (2) identify unusual observations and re-check them; (3) review each group to ensure that matching was conducted according to protocol; (4) review groups with fewer than 3 study participants; and (5) check for consistency of data entry formats.

Participants exhaled directly into two Tedlar® bags or other film bags. The volume of Bag 1 was 0.5 L (SKC Inc. Cat #232-01) and the volume of Bag 2 was 1 L (SKC Inc. Cat #232-02). Each Tedlar® Bag or other film bag was flushed at least 3 times with ultra-high purity nitrogen (Part #NI UHP300, Airgas, US) before use to remove residual contaminants from the manufacturer. Participants were instructed to take a deep inhalation and exhale ˜150-300 mL of breath into the 0.5 L bag (about half full). Immediately, the participant inflated the 1 L bag (Bag 2) using the rest of the exhaled breath. All collected breath samples were delivered at room temperature to the research lab for VOC analysis within 2 hours after collection (whenever possible). The Tedlar bags were measured within 24 hours of breath by the lab in 166 (74%) patients. All but 3 bags were read within 24 hours of lab acquisition. Only data collected from Bag 2 were used for the analyses.

Clean and humidified air was injected into a subset of bags to evaluate measurement background. Volatile organic compounds (VOCs) in the exhaled breath were analyzed using thermal desorption (TD) and gas chromatography-mass spectrometry (GC-MS). A multiple channel thermal desorption system (UNITY-xr™) with an auto-sampler (CIA Advantage-xr™ both from Markes International, Inc., UK) was used to sample 100 mL of exhaled breath from each of the Tedlar® bags or other film bags at a flow rate of 50 mL/min and flow path temperature of 150° C. Helium was used as the carrier gas at a constant pressure of 5 Pounds per Square Inch (PSI); the sample was directly injected from the TD unit into the gas chromatograph for analysis. Chromatographic analysis was performed using a Trace GC-Ultra gas chromatograph attached to an ISQ Mass Spectrometer (GC-MS, Thermo Scientific). VOC compounds were separated with a 30 meter column×0.25 millimeter internal diameter and 1.40 μm film thickness (Cat #19915, Rtx-VMS, Restek Corp, U.S). The oven temperature was set on a gradient to achieve optimal separation of the analytes at an initial temperature of 35° C. with 1 min hold; the temperature rate was increased by 5° C./min to reach 100° C. followed by a final temperature ramp of 50° C./min to 240° C.

Thirteen previously reported VOCs, representing different chemical groups, were selected for quantitative analysis; see Table 1 below for a complete description. For each selected chemical, a five-point calibration curve was generated by spiking reagent-grade standards into Tedlar® bags or other film bags in concentrations ranging from 0.390 μg/mL to 4000 μg/mL using methanol as solvent. Exactly 1 μL aliquot of each standard was injected into five different bags filled with 1 L of pure Nitrogen, diluting the concentration of the analyte by 1000×. Five calibration curves for each VOC were generated, and their average slope and intercept were used to quantify concentrations from participant samples. Ten blanks were prepared by inflating Tedlar® bags or other film bags with clean and humidified air. Clean and humidified air was injected into a subset (10%) of bags to evaluate measurement background.

Volatile organic compounds (VOCs) in the exhaled breath were analyzed using thermal desorption (TD) and gas chromatography-mass spectrometry (GC-MS). The laboratory did not receive information about participant case status. A multiple channel thermal desorption system (UNITY-xr™) with an auto-sampler (CIA Advantage-xr™ both from Markes International, Inc., UK) was used to sample 100 mL of exhaled breath from each of the Tedlar bags at a flow rate of 50 mL/min and flow path temperature of 150° C. Helium was used as the carrier gas at a constant pressure of 5 Pounds per Square Inch (PSI); the sample was directly injected from the TD unit into the gas chromatograph for analysis.

Chromatographic analysis was performed using a Trace GC-Ultra gas chromatograph attached to an ISQ Mass Spectrometer (GC-MS, Thermo Scientific). The lowest standard of each VOC was prepared at least five times and injected into the GC-MS. The limit of detection (LOD) for each chemical was calculated by multiplying the standard deviation of those low analytical standard replicates by 3 (LOD=StDev×3). All lab analysts were blinded to study participant's status and information. Standardized procedures were used for performing and documenting lab operations, including sample management (login, registration integrity, life cycle tracking), chain of custody, inventory and storage management.

Thirteen previously reported VOCs, (Table 1) representing different chemical groups, were selected for quantitative analysis. The limit of detection (LOD) for each chemical was calculated according to the methods provided in the supplementary materials.

TABLE 1
List of quantifiable VOCs and associated references

| No. | CAS | [symbol] | Classification | [symbol] |
|---|---|---|---|---|
| 1 | [symbol] | [symbol] - [symbol] | [symbol] | ( [symbol] ) |
| | | ( [symbol] ) | | |
| 2 | [symbol] | [symbol] | [symbol] | ( [symbol] ) |
| 3 | [symbol] | T [symbol] | [symbol] | ( [symbol] ) |
| 4 | [symbol] - [symbol] - [symbol] | 2- [symbol] | [symbol] | ( [symbol] ) |
| 5 | [symbol] - [symbol] - [symbol] | [symbol] | [symbol] | ( [symbol] ) |
| 6 | [symbol] | 3- [symbol] | [symbol] | ( [symbol] ) |
| 7 | [symbol] | 2- [symbol] | [symbol] | ( [symbol] ) |
| 8 | [symbol] | Dod [symbol] | [symbol] | ( [symbol] ) |
| 9 | [symbol] - [symbol] - [symbol] | [symbol] | [symbol] | ( [symbol] ) |
| 10 | [symbol] | Cy [symbol] | [symbol] | ( [symbol] ) |
| 11 | [symbol] - [symbol] - [symbol] | H [symbol] | [symbol] | ( [symbol] ) |
| 12 | [symbol] - [symbol] - [symbol] | [symbol] | [symbol] | ( [symbol] ) |
| 13 | [symbol] | p- [symbol] | [symbol] | ( [symbol] ) |

[symbol]

indicates data missing or illegible when filed

There are 330 study participants who provided breath samples that were analyzed as part of the study. Not all these participants were included in the analysis because some of them were identified by the clinical team as potential cases, but were not confirmed to have S1LC after biopsy results. The controls for these study participants were also not included in the statistical analysis. Below is the inclusion and exclusion report in Table 2.

TABLE 2
Summary of exclusion reason

| Exclusion reason | Number excluded |
|---|---|
| Other | 21 |
| Stage 2 Lung Cancer | 17 |
| Stage 4 Lung Cancer | 12 |
| Stage 3 Lung Cancer | 6 |
| Carcinoid | 6 |
| Infection | 3 |
| Total | 65 |

TABLE 3
Exclusion reasons for “other” category for potential case

| Exclusion reason | Number excluded |
|---|---|
| benign | 1 |
| breast primary | 1 |
| clean margins from first vats | 1 |
| fibrosis | 1 |
| fungal | 2 |
| granulomas | 3 |
| invasive ini1, smareb1 | 1 |
| lab didn't run sample in time and stage two | 1 |
| met. Renal | 1 |
| neuroendocrine tumor | 1 |
| not cancer | 5 |
| not cancer, histoplasmosis | 1 |
| not lung cancer, probable esophageal cancer | 1 |
| pulmonary infract | 1 |
| Total | 21 |

Since many VOCs were below the limit of detection (LOD) for a large percentage of observations, only four VOCs with less than 10% data below the LOD were used in analyses. Each concentration was log₁₀-transformed. Additional models were fit with each individual VOC being above/below the LOD as a predictor and S1LC as an outcome using univariate logistic regression analysis. A total cohort of 300 individuals was planned: 240 individuals with lung nodules suspicious for possible lung cancer, 30 long-term smokers, and 30 non-smokers. With the overall prevalence of disease of 56.67%, the total sample size of 300 yielded at least 90% power to estimate sensitivity with a 95% confidence interval of ±0.09 at an expected sensitivity of 0.90 and at least 90% power to estimate specificity with a 95% confidence interval of ±0.11 at an expected specificity of 0.90. Power analysis was conducted utilizing R (Vienna, Austria). Following the study analytic protocol, the first 30 groups of matched cases and controls, determined by case enrollment time, were used for training and the last 58 groups were used for testing. Larger proportion of the data was selected for testing to illustrate the higher robustness of the predictions. Analyses were conducted by combining the two types of controls, whenever they were both available.

Each model was fit to the training data, and then applied to: (i) the testing; and (ii) the combined testing and training data. All analyses were performed in the R statistical software. To detect statistically significant differences between VOC breath concentrations in S1LC and controls, two sample unpaired t-tests, which lose some power, but ensure that results are generalizable to the population, were performed using the R function t.test( ). Classification tests using thresholds of the statistically significant VOC were developed based on the 10^{th}, 25^{th} and 50^{th} percentiles of VOC concentrations in the training data of controls. Univariate and multivariate forward selection logistic regression models were fit using the glm( ) function in R. Forward selection was used to identify the combination of most predictive VOCs. Selection of VOCs were based on the improvement in the receiver operating characteristic area under the curve (AUC) in the training data, where at each stage the VOC with the highest AUC in the training data was incorporated into the model. For each selected model the AUC on the test data was computed. Missing observations were excluded in each candidate model when individual VOCs were below the LOD.

Breath samples were collected and analyzed on all study participants who were likely to have S1LC according to the biopsy protocol. However, the breath sample was taken before the biopsy was performed to mitigate the potential effects of sedation and biopsy procedure on the breath VOCs. Among these potential S1LC cases only some had biopsy-confirmed S1LC. There are 157 potential cases in the data (study participants who were likely to have lung cancer before biopsy). Out of these, 65 potential cases (41.4%) did not have biopsy-confirmed S1LC and were excluded from the analysis. All excluded cases had a valid exclusion reason recorded in RedCap. Table 4 summarizes the exclusion criteria for these potential cases after biopsy results. The specific reasons for excluding potential cases labeled “Other” in Table 4 are provided in Table 5. The category “Other” was used in Table 4 because the reasons for non-inclusion listed in Table 5 are rare. Most potential cases were excluded because biopsy results were negative (person did not have biopsy-confirmed S1LC, even though they were considered likely to have S1LC before the biopsy was conducted). For the purpose of this analysis the matched and housemate control data associated with the cases that met the exclusion criteria have also been removed from the analysis. These data exist for some study participants, but was not included in the analysis.

TABLE 4
Number of control types available for each case

| Control type | Number available |
|---|---|
| No Controls | 2 |
| Housemate Control Only | 2 |
| Matched Control Only | 39 |
| Both Controls | 49 |
| Total | 92 |

After the exclusion of groups that did not have a biopsy-confirmed S1LC case, there were 231 study participants left (cases and controls). These data include a total of 92 cases with 51 control housemates and 88 matched controls. The number and type of controls are displayed for these 92 cases in Table 6.

From these data four patients with biopsy-confirmed S1LC were further excluded. Out of these four patients 2 did not have either matched or housemate control data. The other 2 cases had only housemate control but not matched control data. Data for these groups were excluded from the analysis. These exclusions were applied to avoid groups that are not balanced on covariates.

Data analysis is conducted only for groups of study participants that contained a patient with biopsy-confirmed S1LC. For this analysis, only 88 groups with a case who had least one available matched control were used. These data are referred to as “included groups”. Among the included groups, 39 groups had only one matched control and no housemate control and 49 had both matched control and housemate controls; see Table 6 for more details.

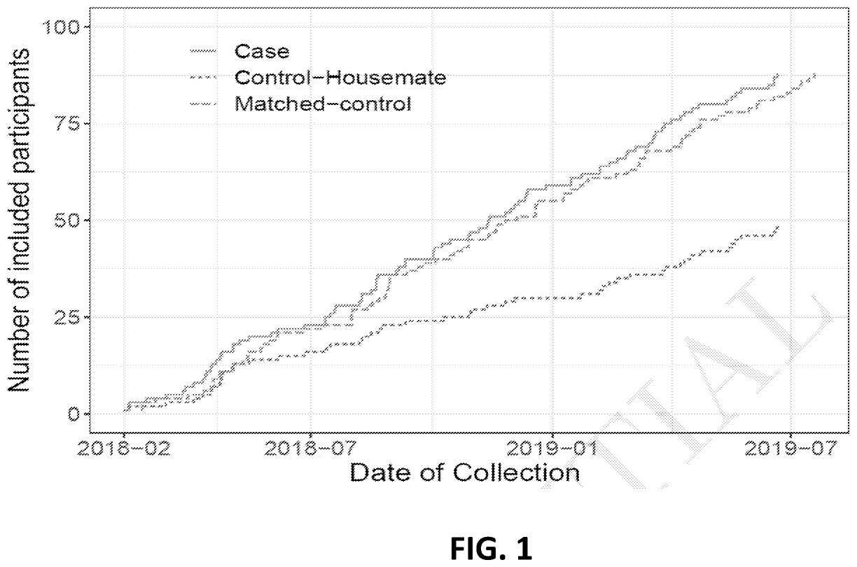

FIG. 1 illustrates a graphical view of an enrollment graph for the study as a function of time. Each line represents the cumulative enrollment by participant type (case=solid line, matched control=dotted line, and housemate control=dashed line). For example, by January 2019 there were 55 enrolled biopsy-confirmed S1LC cases in the study.

There are 330 total number of study participants, which included 157 potential cases (patients who were identified by the clinical team as potential cases before the biopsy). Out of the 157 potential cases, 65 (41.4%) were excluded from the analysis. Most potential cases were excluded from the study because biopsy results did not confirm the S1LC diagnosis; see Table 2. Matching control and housemate control data associated with the cases that were excluded were also removed from the analysis. The data used in this analysis has 225 participants, which includes a total of 88 cases with at least one available matched control.

According to the pre-specified analysis protocol, data were split into training (for biomarker discovery and model exploration) and testing (for validation of biomarkers and models). The first 30 groups and their controls were used for training and the remaining 58 groups were used for testing.

TABLE 5
Demographics table for included data

| | | | Matched | | Housemate | | |
|---|---|---|---|---|---|---|---|
| | | | control | | Control | | |
| | Case | | (N = 88, | | (N = 49, | | |
| | (N = 88) | | type 1) | | type 2) | | p-value |
| Age (mean (SD)) | 67.85 | (9.28) | 67.91 | (9.81) | 63.13 | (14.15) | 0.025 |
| BMI (mean (SD)) | 27.61 | (6.11) | 28.68 | (5.60) | 28.35 | (4.81) | 0.446 |
| Smoking history (no. (%)) | | | | | | | <0.001 |
| Never | 16 | (18.2) | 16 | (18.2) | 31 | (63.3) | |
| Current | 14 | (15.9) | 14 | (15.9) | 3 | (6.1) | |
| Former | 58 | (65.9) | 58 | (65.9) | 15 | (30.6) | |
| Race (no. (%)) | | | | | | | 0.273 |
| White | 63 | (71.6) | 62 | (70.5) | 38 | (77.6) | |
| Black | 17 | (19.3) | 19 | (21.6) | 3 | (6.1) | |
| Asian/Pacific Islander | 6 | (6.8) | 6 | (6.8) | 6 | (12.2) | |
| Other | 2 | (2.3) | 1 | (1.1) | 2 | (4.1) | |
| Female sex (no. (%)) | 52 | (59.1) | 52 | (59.1) | 24 | (49.0) | 0.450 |
| No history of family | 56 | (63.6) | 85 | (96.6) | 39 | (79.6) | <0.001 |
| cancer (no. (%)) | | | | | | | |
| No kidney disease (no. (%)) | 72 | (81.8) | 80 | (90.9) | 47 | (95.9) | 0.030 |
| No diabetes (no. (%)) | 70 | (79.5) | 70 | (79.5) | 41 | (83.7) | 0.813 |
| No liver disease (no. (%)) | 86 | (97.7) | 82 | (93.2) | 47 | (95.9) | 0.340 |
| No alcohol use (no. (%)) | 37 | (42.0) | 29 | (33.0) | 20 | (40.8) | 0.423 |

The demographic and behavioral summaries for the study participants in the 88 analyzed groups (case and at least one available matched control) are presented in Table 5. Details are further provided by the three study participant types (case, matched control, housemate control). Table 6 provides the demographic and behavioral information separated by training and testing data sets. For each subject, two bags of exhaled breath were collected consecutively during one forceful exhalation process. Bag 1 (diluted) had a volume of 0.5 liters and was used to collect the first air exhaled (tidal volume), which is thought to represent the normal exhalation process. Bag 2 (alveolar) had a volume of 1.0 liter and was used to collect the expiratory reserve volume (the gas mixture coming from the dead space of the bronchial tree and the alveolar gas exchange space of the lungs). The air from each bag was injected into a gas chromatograph (GC-MS), which separated the different compounds in the exhaled air into a series of “peaks”. Each peak was associated with a distinct VOC.

To convert an original GC-MS peak area result (unitless) to a concentration value in the sample (mass of compound per volume of air), a calibration curve was constructed for each of the 13 quantifiable VOC compounds described in Section 4.7.3. A calibration curve was obtained by serially diluting a chemical standard to obtain at least five different and known concentrations, which are plotted along the x-axis. These known concentrations are injected into the GC-MS and the resulting peaks are plotted along the y-axis. Each calibration curve was compound specific. This provided the mapping (calibration) of VOC peak areas to concentrations measurements for Bags 1 and 2.

TABLE 6
Demographics table for S1LC patients in included data

| | C [symbol] | | | | |
|---|---|---|---|---|---|
| | Train | | Test | | p |
| n | [symbol] 0 | | [symbol] 8 | | |
| age (mean (SD)) | 6 [symbol] .68 | ( [symbol] .13) | [symbol] . [symbol] | ( [symbol] .23) | 0.114 |
| [symbol] (mean (SD)) | 27.33 | ( [symbol] .37) | 27.70 | ( [symbol] .0 [symbol] ) | 0.761 |
| smokehistory (%) | | | | | 0.747 |
| Never | [symbol] | (10.7) | 11 | (19.0) | |
| Current | 6 | (20.0) | 8 | (13.8) | |
| Former | 19 | ( [symbol] . [symbol] ) | 39 | (67.2) | |
| race (%) | | | | | 0.197 |
| White | 19 | (63.3) | 44 | (7 [symbol] .9) | |
| Black | 7 | (23. [symbol] ) | 10 | (17.2) | |
| Asian/Pacific Islander | 4 | (13.3) | 2 | (3.4) | |
| Native American/Alaskan Native | 0 | (0.0) | 0 | (0.0) | |
| Other | 0 | (0.0) | 2 | (3.4) | |
| sex = Femal (%) | 18 | ( [symbol] 0.0) | 34 | ( [symbol] 8.0) | 1.000 |
| cancerhistory = No (%) | 20 | ( [symbol] .7) | 3 [symbol] | (62.1) | 0.848 |
| kidneydisease = No (%) | 24 | ( [symbol] .0) | 48 | (82.8) | 0.979 |
| diabetes = No (%) | 22 | (73. [symbol] ) | 48 | (82.8) | 0.447 |
| liverdisease = No (%) | 29 | ( [symbol] .7) | 57 | (98.3) | 1.000 |
| alcohol = No (%) | 15 | ( [symbol] .0) | 22 | (37.9) | 0.3 [symbol] 0 |

[symbol]

indicates data missing or illegible when filed

The first step is to compare the consistency of VOC quantification in the two bags. Note: bag comparison results are based on the analyzed data only, which included 225 study participants (88 cases, 88 matched controls, and 49 housemate controls). As both measures are highly right skewed, the log₁₀ (peak area) and log₁₀ (concentration) were used instead.

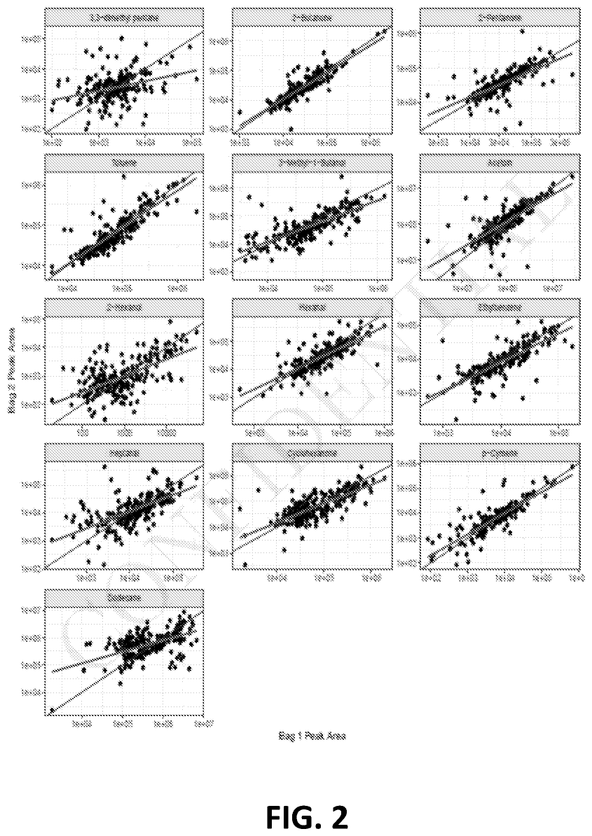

Results indicate that for most compounds, the VOC peak areas measurements for the two bags are strongly correlated; see Table 7 and FIG. 2. These results are based on larger sample sizes than the corresponding results for concentrations, which require both bag measurements to be above the limit of detection. The columns labeled “n” in Table 7 provide the number of study participants who had both bag measurements by VOC. For VOCs with concentrations above the limit of detection in more than 100 bag pairs there is a good agreement between the correlation of VOC peaks and concentrations.

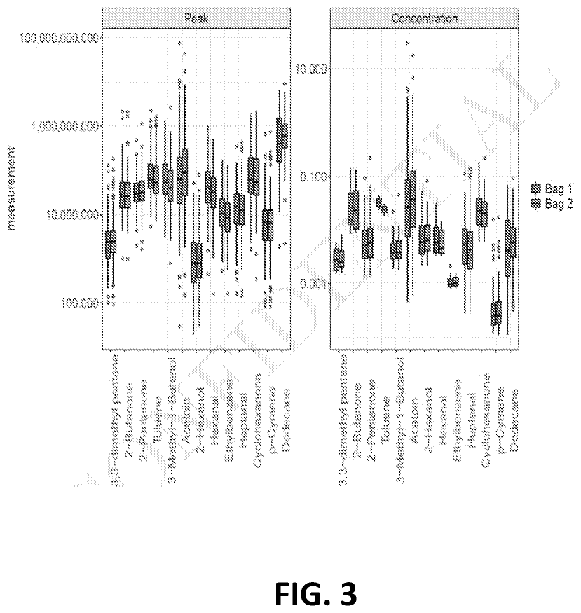

FIG. 3 provides the distributions of VOC peak areas (left panel) and concentrations (right panel) in Bags 1 (dark grey) and 2 (light grey) separated by compound (x-axis). For 2-Pentanone, Acetoin, and Dodecane the log-peak areas and concentrations was smaller on average in Bag 1 than in Bag 2. However, for the other VOCs measurements tended to be on average similar in the two Bags or even larger in Bag 1. Table 9 provides the results of paired t-tests for the null hypothesis of no difference in the mean log₁₀ peak areas and concentrations between Bags 1 and 2. For log₁₀ peak areas there was a statistically significant difference for 3-Methyl-1-Butanol (p-values 0.001, larger values in Bag 1), Acetoin (p-values 0.001, smaller values in Bag 1), Dodecane (p-value=0.005, smaller values in Bag 1), Ethylbenzene (p-values 0.001, larger values in Bag 1), Hexanal (p-values 0.001, larger values in Bag 1), and Toluene (p-values 0.001, larger values in Bag 1). For log₁₀ concentrations there was a statistically significant difference for 2-Pentanone (p-value=0.002, smaller values in Bag 1), Acetoin (p-values 0.001, smaller values in Bag 1), Cyclohexanone (p-value=0.018, larger values in Bag 1), and Dodecane (p-value=0.009, smaller values in Bag 1). The difference in testing results between log peak areas and concentrations can be attributed to the large number of missing concentrations that are below the limit of detection.

FIG. 2 illustrates graphical views of scatterplots of log₁₀ (peak) for Bag 1 (x-axis) versus Bag 2 (y-axis). Dark grey: regression line; light grey: identity line; axis labels: displayed on the original scale. FIG. 3 illustrates graphical views of boxplots of peak area (left panel) and concentrations (right panel) on log₁₀ scale for Bag 1 (dark grey) and Bag 2 (light grey). The y-axes labels are displayed on the original scale. The peak data are unitless and the concentrations are expressed in μg/L.

TABLE 7
Bag 1 vs. Bag 2. In each pairwise complete comparison values with
missing or undetected peak or concentration in either Bag 1 or
Bag 2 were excluded from analysis. The number of samples used
to compute correlation is recorded in column n samples.

| | Peak | | | | Concentrations | | | |
|---|---|---|---|---|---|---|---|---|
| Compound | [symbol] | [symbol] | [symbol] | [symbol] | [symbol] | [symbol] | [symbol] | [symbol] |
| 3,3- [symbol] | 0.232 | 0. [symbol] 1 | 0. [symbol] 1 | 20 [symbol] | 2 [symbol] | NA | NA | 1 |
| 2- [symbol] | 0. [symbol] | 0. [symbol] | 0. [symbol] | 215 | 0.991 | 0.9 [symbol] | 1.000 | 4 |
| 2-P [symbol] | 0. [symbol] | 0.70 [symbol] | 0.701 | 215 | 0.334 | 0. [symbol] | 0.8 [symbol] | [symbol] |
| T [symbol] | 0. [symbol] | 0.807 | 0.870 | 215 | NA | NA | NA | 1 |
| [symbol] - [symbol] -1- [symbol] | 0.112 | 0.7 [symbol] | 0.77 [symbol] | 215 | −0. [symbol] | [symbol] | [symbol] | [symbol] |
| Ac [symbol] | 0. [symbol] | 0.7 [symbol] | 0.7 [symbol] | 21 [symbol] | 0. [symbol] | 0. [symbol] | 0.787 | 205 |
| 2-H [symbol] | 0. [symbol] | 0. [symbol] | 0. [symbol] | 214 | 0.411 | 0. [symbol] | 0. [symbol] | 36 |
| He [symbol] | 0. [symbol] | 0.7 [symbol] | 0.813 | 210 | 0.2 [symbol] 2 | 0.247 | 0.2 [symbol] 7 | 9 |
| [symbol] | 0. [symbol] | 0. [symbol] | 0. [symbol] | 210 | 1.000 | 0. [symbol] | 1.000 | [symbol] |
| H [symbol] | 0. [symbol] | 0. [symbol] | 0. [symbol] | 20 [symbol] | 0. [symbol] | 0. [symbol] | 0. [symbol] | [symbol] |
| Cy [symbol] | 0. [symbol] | 0.7 [symbol] | 0.7 [symbol] | 21 [symbol] | 0. [symbol] | 0. [symbol] | 0. [symbol] | [symbol] |
| [symbol] -C [symbol] | 0. [symbol] | 0. [symbol] | 0. [symbol] | 211 | 0. [symbol] | 0. [symbol] | 0.7 [symbol] | [symbol] |
| D [symbol] | 0. [symbol] | 0. [symbol] | 0. [symbol] | 213 | 0. [symbol] | 0. [symbol] | 0. [symbol] | 207 |

[symbol]

indicates data missing or illegible when filed

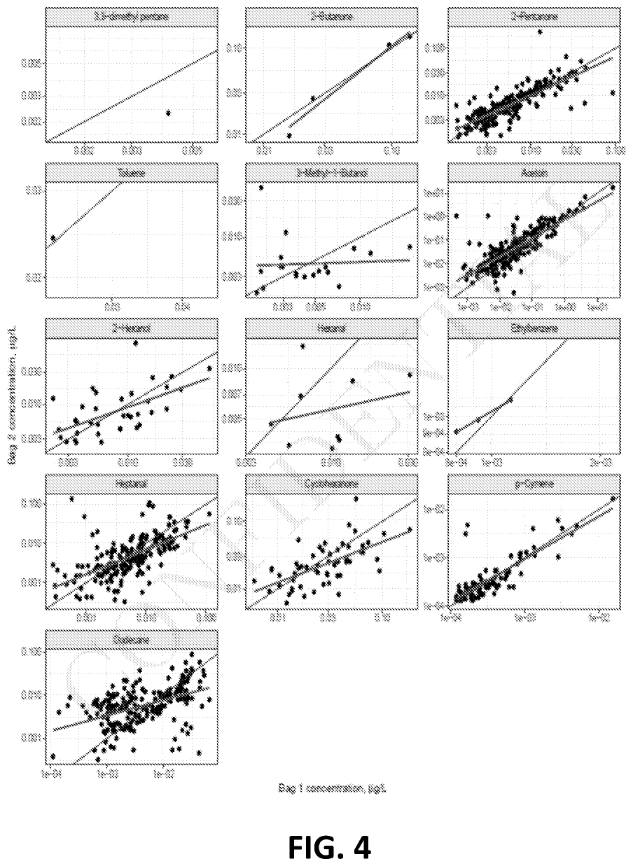

The association between the measurements in the two bags was also quantified using a linear model regression for Bag 2 (y, outcome) versus Bag 1 (x, regressor) based on log₁₀ peak areas and concentrations, respectively. Table 9 provides summaries of these regressions, where: (1) the columns labeled “Estimate” provide the point estimate for the slope of the regression; (2) the column labeled “p-value” is the p-value for testing the null hypothesis of no association between measurements in Bags 1 and 2; (3) the columns labeled “lower CL” and “upper CL” are the lower and upper limits of the 95% confidence intervals for the participants who had both bag measurements. Results indicate that there is strong evidence that the log peak area measurements in the two Bags are strongly statistically associated for all quantifiable compounds peak data, where 2-Butanone, Toluene have the slope estimates greater than 0.9 and Ethylbenzene, p-Cymene greater than 0.8. Scatterplots of Bag 1 (x-axis) versus Bag 2 (y-axis) measurements are shown in FIG. 2 for peak area and FIG. 4 for concentrations. The regression line is shown in dark grey and the 45° (identity) line is shown in light grey. Data are plotted on the log scale, but labels are shown on the original scale. FIG. 2 indicates strong associations between the peak area measurements in Bags 1 and 2 for most compounds and were used for quality control purposes.

FIG. 4 illustrates graphical views of scatterplots of log₁₀ (concentration) for Bag 1 (x-axis) versus Bag 2 (y-axis). Dark grey: regression line; light grey: the identity line; axis labels: displayed on the original scale.

TABLE 8
Paired t-test of no difference between Bag 1 and Bag 2 using logio
peak areas and concentrations. P-values and the lower and upper
confidence limits of the 95% confidence intervals are provided

| | Peak | | | Concentration | | |
|---|---|---|---|---|---|---|
| | | Lower | Upper | | Lower | Upper |
| Compound | p-value | CL | CL | p-value | CL | CL |
| 3,3-dimethyl pentane | 0.500 | −0.103 | 0.051 | NA | NA | NA |
| 2-Butanone | 0.164 | −0.008 | 0.048 | 0.621 | −0.165 | 0.233 |
| 2-Pentanone | 0.122 | −0.005 | 0.008 | 0.002 | −0.086 | −0.020 |
| Toluene | 0.004 | 0.015 | 0.078 | NA | NA | NA |
| 3-Methyl-1-Butanol | 0.000 | 0.066 | 0.164 | 0.834 | −0.205 | 0.167 |
| Acetoin | 0.000 | −0.274 | −0.106 | 0.000 | −0.279 | −0.142 |
| 2-Hexanol | 0.378 | −0.109 | 0.042 | 0.955 | −0.108 | 0.102 |
| Ethylbenzene | 0.000 | 0.049 | 0.133 | 0.305 | −0.104 | 0.054 |
| Heptanal | 0.830 | −0.059 | 0.073 | 0.102 | −0.011 | 0.119 |
| Cyclohexanone | 0.079 | −0.006 | 0.099 | 0.018 | 0.016 | 0.159 |
| p-Cy [symbol] | 0.147 | −0.079 | 0.012 | 0.399 | −0.085 | 0.034 |
| Dodecane | 0.005 | −0.170 | −0.030 | 0.009 | −0.176 | −0.026 |

[symbol]

indicates data missing or illegible when filed

The fewer data points in FIG. 2 compared to FIG. 4 is due to the fact that many concentrations were below the limit of detection. For this reason the estimates of the slope parameters for the regression of log concentrations in Bag 2 versus Bag 1 tended to be smaller than for log peaks. For log concentrations only 2-Butanone, 2-Pentanone, p-Cymene had the slope estimates larger than 0.8.

According to the study design, each study participant started exhaling in Bag 1 (diluted), and continued exhaling into Bag 2 (alveolar), which was assumed to collect deeper air from the lungs. Comparison of Bags 1 and 2 peak area and concentration measurements indicates that there are strong correlation between the measurements in the two bags; see Table 7, FIG. 2 and FIG. 4. For some compounds there are statistically significant differences between Bag 1 and 2 measurements. For log₁₀ peak areas some VOC measurements are higher on average in Bag 1 and some a higher in Bag 2. For log₁₀ concentrations there were either no statistically significant differences between the two Bags or measurements were lower on average in Bag 1. These differences can be attributed to the large number of missing concentrations (below limit of detection) for many VOCs. For the purposes of data analysis only Bag 2 data was used.

TABLE 9
Bag similarity: linear fit results and 95 percent confidence intervals.
Bag 1 (x) vs. Bag 2 (y), each were logio transformed.

| | Peak | | | | | Concentrations | | | | |
|---|---|---|---|---|---|---|---|---|---|---|
| Compound | [symbol] | [symbol] | [symbol] | [symbol] | N | [symbol] | [symbol] | [symbol] | [symbol] | N |
| 3,3- [symbol] | 0. [symbol] | <0.00 [symbol] | 0. [symbol] 1 | [symbol] | [symbol] | NA | NA | NA | NA | [symbol] |
| 2- [symbol] | 0. [symbol] | <0. [symbol] | 0. [symbol] | 0. [symbol] | 215 | 1. [symbol] | 0. [symbol] | 0. [symbol] | 1. [symbol] | [symbol] |
| 2- | 0. [symbol] | <0. [symbol] | 0. [symbol] | 0. [symbol] | 215 | 0. [symbol] | <0. [symbol] | 0. [symbol] | 0. [symbol] | [symbol] |
| T [symbol] | 0. [symbol] | <0. [symbol] | 0. [symbol] | 0. [symbol] | 215 | NA | NA | NA | NA | [symbol] |
| [symbol] - [symbol] -1- [symbol] | 0. [symbol] | <0. [symbol] | 0. [symbol] | 0. [symbol] | 215 | 0. [symbol] | 0. [symbol] | −0. [symbol] | 0. [symbol] | [symbol] |
| Ac [symbol] | 0. [symbol] | <0. [symbol] | 0. [symbol] | 0. [symbol] | 21 [symbol] | 0. [symbol] | <0.001 | 0. [symbol] | 0. [symbol] | [symbol] |
| 2-H [symbol] | 0. [symbol] | <0. [symbol] | 0. [symbol] | 0. [symbol] | 21 [symbol] | 0. [symbol] | <0.001 | 0. [symbol] | 0. [symbol] | [symbol] |
| He [symbol] | 0. [symbol] | <0. [symbol] | 0. [symbol] | 0. [symbol] | 21 [symbol] | 0. [symbol] | 0. [symbol] | 0. [symbol] | 0. [symbol] | [symbol] |
| [symbol] | 0. [symbol] | <0. [symbol] | 0. [symbol] | 0. [symbol] | 21 [symbol] | 0. [symbol] | 0. [symbol] | 0. [symbol] | 0. [symbol] | [symbol] |
| H [symbol] | 0. [symbol] | <0. [symbol] | 0. [symbol] | 0. [symbol] | 2 [symbol] | 0. [symbol] | <0.001 | 0. [symbol] | 0. [symbol] | [symbol] |
| Cy [symbol] | 0. [symbol] | <0. [symbol] | 0. [symbol] | 0. [symbol] | 2 [symbol] | 0. [symbol] | <0.001 | 0. [symbol] | 0. [symbol] | [symbol] |
| [symbol] -C [symbol] | 0. [symbol] | <0. [symbol] | 0. [symbol] | 0. [symbol] | 2 [symbol] | 0. [symbol] | <0.001 | 0. [symbol] | [symbol] | [symbol] |
| D [symbol] | 0. [symbol] | <0. [symbol] | 0. [symbol] | 0. [symbol] | [symbol] | 0. [symbol] | <0.001 | 0. [symbol] | 0. [symbol] | [symbol] |

[symbol]

indicates data missing or illegible when filed

TABLE 10
Number of undetected compounds by bag information (included
participants data with at least one matched control)

| | Concentration | | |
|---|---|---|---|
| | | Bag 1, | Bag 2, |
| Compound | | n = 221 | n = 225 |

| | | | | |
|---|---|---|---|---|
| 3,3-dimethyl pentane | 217 | (98.2%) | 219 | (97.3%) |
| 2-Butanone | 214 | (96.8%) | 218 | (96.9%) |
| 2-Pentanone | 25 | (11.3%) | 17 | (7.6%) |
| Toluene | 219 | (99.1%) | 222 | (98.7%) |
| 3-Methyl-1-Butanol | 183 | (82.8%) | 200 | (88.9%) |
| Acetoin | 12 | (5.4%) | 7 | (3.1%) |
| 2-Hexanol | 170 | (76.9%) | 172 | (76.4%) |
| Hexanal | 200 | (90.5%) | 209 | (92.9%) |
| Ethylbenzene | 213 | (96.4%) | 217 | (96.4%) |
| Heptanal | 25 | (11.3%) | 19 | (8.4%) |
| Cyclohexanone | 142 | (64.3%) | 148 | (65.8%) |
| p-Cymene | 121 | (54.8%) | 126 | (56.0%) |
| Dodecane | 11 | (5.0%) | 4 | (1.8%) |

Quantifiable compounds were not detected for some study participants. The missing (below limit of detection) concentrations by VOC and collection bag are presented in Table 10. Here missing concentration values include both missing peak values, which did not produce a concentration value after calibration, and peak values which corresponded to a VOC concentration value that was considered below the limit of detection. There were 4 (Control-Housemate: N=1, Matched-control: N=3) study participants in the test data set with missing Bag 1 measurement. These study participants were removed from the Bag 1 versus 2 analysis, but were kept in the predictive modeling analysis.

TABLE 11
Number of undetected concentrations in Bag 2

| | Bag 2 | | | | | | | |
|---|---|---|---|---|---|---|---|---|
| | Train | | | | Test | | | |
| | Case, | | Control, | | Case, | | Control, | |
| Compound | n = 30 | | n = 51 | | n = 58 | | n = 86 | |
| 2-Pentanone | 2 | (6%) | 3 | (6%) | 3 | (6%) | 9 | (10%) |
| Dodecane | 1 | (4%) | 0 | (0%) | 2 | (4%) | 1 | (2%) |
| Acetoin | 2 | (6%) | 2 | (4%) | 1 | (2%) | 2 | (2%) |
| Cyclohexanone | 6 | (20%) | 15 | (30%) | 52 | (90%) | 75 | (88%) |
| Heptanal | 7 | (24%) | 6 | (12%) | 2 | (4%) | 4 | (4%) |
| p-Cymene | 19 | (64%) | 10 | (38%) | 41 | (70%) | 47 | (54%) |
| 2-Hexanol | 18 | (60%) | 25 | (50%) | 51 | (88%) | 78 | (90%) |
| 3-Methyl-1-Butanol | 26 | (86%) | 38 | (74%) | 56 | (96%) | 80 | (94%) |
| Hexanal | 27 | (90%) | 43 | (84%) | 56 | (96%) | 83 | (96%) |
| 2-Butanone | 28 | (94%) | 48 | (94%) | 57 | (98%) | 85 | (98%) |
| 3,3-dimethyl | 28 | (94%) | 48 | (94%) | 58 | (100%) | 85 | (98%) |
| pentane | | | | | | | | |
| Toluene | 28 | (94%) | 50 | (98%) | 58 | (100%) | 86 | (100%) |
| Ethylbenzene | 28 | (94%) | 48 | (94%) | 56 | (9 [symbol] %) | 85 | (98%) |

[symbol]

indicates data missing or illegible when filed

Table 11 further lists the number of case and control study participants in the training and testing data with missing quantifiable peaks and concentrations, respectively. Results indicate that the individual VOC limit of detection and percent missingness depends on the compound type both for peaks and concentrations. There is also a bag effect for peak areas, with fewer missing peak areas in Bag 2 (with the exception of Ethylbenzene). For concentrations with lower percent missingness (Dodecane, Acetoin, 2-Pentanone, Heptanal) the percent missing observations was lower in Bag 2. For concentrations with higher percent missingness the difference between bags was less clear.

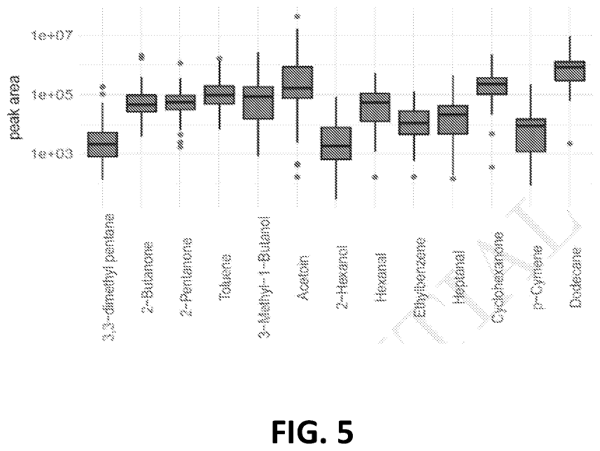

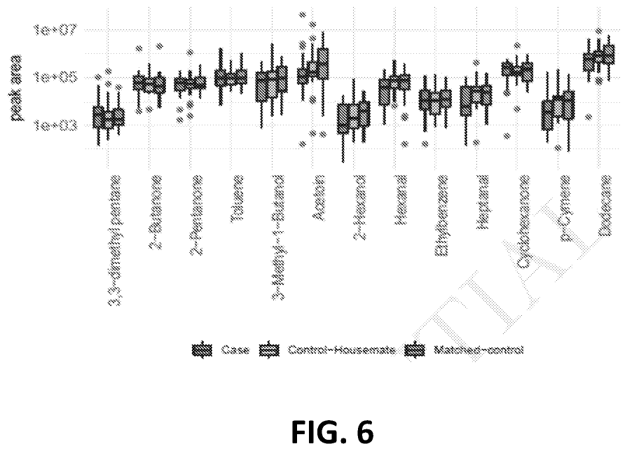

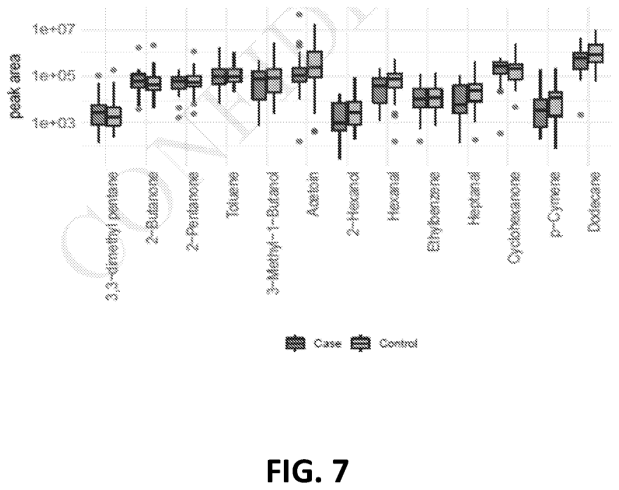

The quantifiable VOC peak area obtained from Bag 2 (alveolar) in the training data is examined. FIG. 5 displays the boxplot of log₁₀ (peak) area for cases and controls combined. The x-axis is on the original scale even though data were log₁₀-transformed. FIG. 6 displays the same data as FIG. 5, but boxplots are separated by cases (dark grey), housemate controls (light grey) and matched controls (grey). A visual inspection of the data suggests that Acetoin, 2-Hexanal, Hexanal, Heptanal, p-Cymene and Dodecane exhibit differences in the distribution of log₁₀ peak areas between cases and controls in the training data. For all of these VOCs, cases tend to have on average lower, not higher, log₁₀ peak areas than controls. FIG. 7 displays the same data as FIG. 6, with cases shown in dark grey and controls (combined housemate and matched controls) shown in light grey.

FIG. 5 illustrates graphical views of boxplots of log₁₀ (peak) for quantifiable VOCs. The x-axis are the compounds and the y-axis labels are displayed on the original scale while the data were log₁₀ transformed. VOC peaks in S1LC cases tend to be lower than in controls, which contradicts currently published literature Acetoin, 2-Hexanal, Hexanal, Heptanal, p-Cymene and Dodecane exhibit visual differences in the distribution of log₁₀ peak areas between cases and controls in the training data.

The overall goal of the project is to identify individual or VOC combinations that discriminate S1LC patients from controls. The first step was to conduct forward selection based on logistic regression on the training data, regressing on the case/control status. The ideas is to select the combination of variables with the highest predictive performance as measured by the area under the receiver operating characteristic (AUC) curve in the training data set. The second step is to apply and evaluate these models on the test data set. A control is defined as a study participant in the “included data” subset who does not have cancer (either control housemate or matched control). For each compound the missing observations were removed in all models that contained that compound.

FIG. 6 illustrates graphical views of boxplots of log₁₀(peak) for quantifiable VOCs separated by cases (dark grey), housemate controls (light grey), and matched controls (grey). The x-axis are the compounds and the y-axis labels are displayed on the original scale even though the data were log₁₀ transformed.

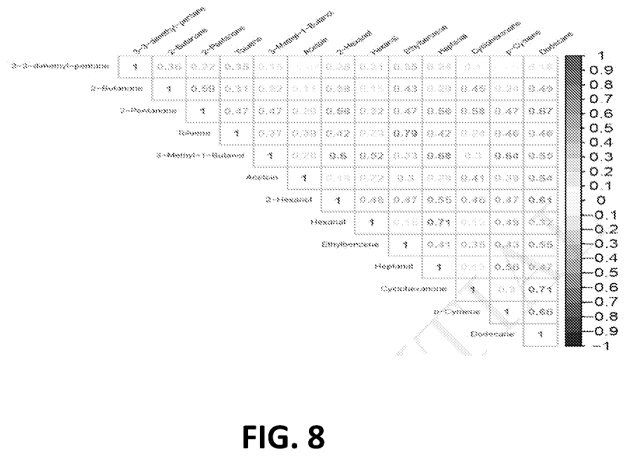

FIG. 7 illustrates graphical views of boxplots of log₁₀(peak) for quantifiable VOCs separated by cases (dark grey), housemate and matched controls combined (light grey). The x-axis are the compounds and the y-axis labels are displayed on the original scale even though the data were log₁₀ transformed. FIG. 8 illustrates an info graphical view of correlation among VOC log₁₀ (peaks).

Pairs of VOCs with high correlations between log peak area measurements may not improve the predictive performance of models using only one of the VOCs in the pair. This is due to the overlap in information between the two VOCs in the pair. On the contrary, pairs of VOCs with low correlations are good candidates for jointly improving prediction. In this data set, many VOC pairs have highly correlated log peaks; see FIG. 8. For example, Toluene has a correlation of 0.79 with Ethylbenzene and Hexanal has a correlation of 0.71 with Heptanal. In contrast, Acetoin has lower correlations with all quantifiable compounds, with a maximum correlation of 0.54 with Dodecane.

The performance of each VOC (log peak area) in a univariate model is examined, that is, using each VOC as a single predictor of lung cancer. Table 12 ranks predictive performance of each compound. Based on the training AUC results, p-Cymene, Heptanal, Acetoin are the top 3 VOCs in terms of S1LC case prediction performance. Table 12 also shows that the top individual predictors ranked by test AUC are Acetoin (test AUC 0.648), p-Cymene (test AUC 0.612) and 2-Butanone (test AUC 0.61).

Table 12 displays the results of the forward selection procedure, where each VOC is added in the predictive model based on the maximum AUC criteria in the training set. The model with maximum test AUC included p-Cymene and 2-Butanone (test AUC 0.669). The second best performing model included p-Cymene, 2-Butanone, Heptanal, and Acetonin (test AUC 0.620).

TABLE 12
VOCs ranked by the individual prediction performance
of S1LC cases based on log peak area. Ranking
criterion: AUC in the training data set

| | |
|---|---|
| Train | Test |

| | Individual | | Individual | |
|---|---|---|---|---|
| Variable | AUC | N | AUC | N |
| p-Cymene | 0.668 | 81 | 0.612 | 142 |
| Heptanal | 0.655 | 77 | 0.501 | 142 |
| Acetoin | 0.636 | 80 | 0.648 | 143 |
| Hexanal | 0.636 | 79 | 0.480 | 143 |
| 2-Hexanol | 0.603 | 80 | 0.538 | 143 |
| Dodecane | 0.588 | 81 | 0.532 | 142 |
| 3-Mathyl-1-Butanol | 0.582 | 81 | 0.555 | 143 |
| 3-3-dimethyl-pentane | 0.556 | 73 | 0.540 | 143 |
| Cyclohexanone | 0.537 | 81 | 0.488 | 142 |
| Toluene | 0.536 | 81 | 0.553 | 143 |
| 2-Butanone | 0.535 | 81 | 0.610 | 143 |
| Ethylbenzene | 0.524 | 79 | 0.524 | 140 |
| 2-Pentanone | 0.497 | 81 | 0.599 | 143 |

A major practical limitation of the VOC peak-based analysis is that multiple compounds are below the limit of detection; see Tables 10 and 11. For example, the top predictor based on log peak area used p-Cymene (64% missing concentrations in cases/training, 38% missing concentrations in controls/training, 70% missing concentrations in cases/test, and 47% missing concentrations in controls/test) and 2-Butanone (94% missing concentrations in training cases and controls and 98% missing concentrations in test cases and controls). This is a problem because even if the compounds may have discriminatory power, they are generally under the limit of detection of the GC-MS instrument used in the study. The implication is that concentration thresholds with discriminating properties cannot be provided for these compounds.

Therefore, in what follows VOC concentrations with values above the limit of Detection are used.

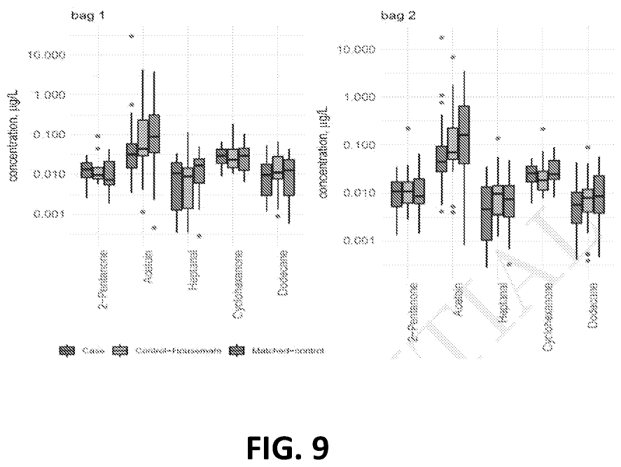

FIG. 9 compares VOC concentrations for training data separated by each control type and the two bags (left panel corresponds to Bag 1 and right panel corresponds to Bag 2). Only compounds with less than 20% missing data (either in Bag 1 or 2) are used in the analysis. Boxplots are shown in dark grey for cases, light grey for housemate controls, and grey for matched controls. For each compound the boxplots are based on a different number of study participants, as missing concentrations were excluded.

TABLE 13
Forward selection models using log peak area for VOCs based
on maximum improvement in AUC in the training data

| | Train | | Test | |
|---|---|---|---|---|
| | Cumulative | | Cumulative | |
| | AUC | N | AUC | N |
| p-Cymene | 0.668 | 81 | 0.612 | 142 |
| 2-Butanone | 0.680 | 81 | 0.66 [symbol] | 142 |
| Heptanal | 0.695 | 77 | 0.577 | 142 |
| Acetoin | 0.717 | 76 | 0.620 | 142 |
| 3-Methyl-1-Butanol | 0.749 | 76 | 0.580 | 142 |
| 3-3-dimethyl-pentane | 0.779 | 71 | 0.558 | 142 |
| Toluene | 0.797 | 71 | 0.531 | 142 |
| 2-Hexanol | 0.804 | 71 | 0.533 | 142 |
| Ethylbenzene | 0.806 | 70 | 0.544 | 140 |
| Hexanal | 0.804 | 70 | 0.535 | 140 |
| 2-Pentanone | 0.803 | 70 | 0.559 | 140 |
| Dodecane | 0.808 | 70 | 0.524 | 140 |
| Cyclohexanone | 0.806 | 70 | 0.506 | 140 |

[symbol]

indicates data missing or illegible when filed

TABLE 14
Correlations of log concentrations of quantifiable VOCs that have at
least 20% concentration measurements above the limit of detection

| | 2-Pentanone | Acetoin | Cyclohexanone | Dodecane | Heptanal |
|---|---|---|---|---|---|
| 2-Pentanone | 1.000 | 0.110 | 0.509 | 0.65 [symbol] | 0.446 |
| Acetoin | 0.110 | 1.000 | 0.074 | 0.374 | 0.369 |
| Cyclohexanone | 0.509 | 0.074 | 1.000 | 0.529 | 0.237 |
| Dodecane | 0.656 | 0.374 | 0.529 | 1.000 | 0.488 |
| Heptanal | 0.446 | 0.369 | 0.237 | 0.488 | 1.000 |

[symbol]

indicates data missing or illegible when filed

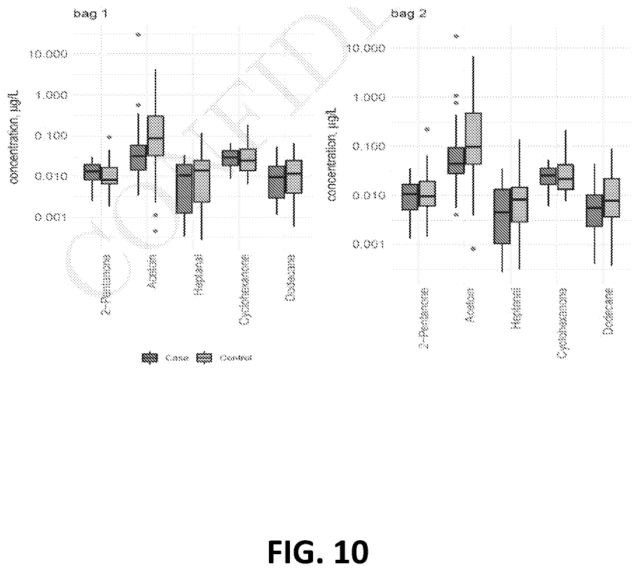

FIG. 10 provides the same information as FIG. 9, but combines housemate and matched control data into a single control category. According to the protocol, only the data obtained from Bag 2 is used. For prediction modeling the housemate and matched controls are combined into one category, as shown in FIG. 10.

Correlations between individual VOC log concentrations (using pairwise complete observations) in the training data are presented in Table 14. Results are consistent with the correlation results for VOC log peak areas; see FIG. 2. Dodecane and 2-Pentanone had highest correlation (correlation 0.656) among all VOC pairs. Acetoin has consistently low correlations with the other quantifiable VOCs shown in Table 14 with the largest correlation in absolute value with Dodecane (correlation 0.374) and Heptanal (correlation 0.369).

FIG. 9 illustrates graphical views of boxplots of log₁₀ (concentrations) for quantifiable VOCs with concentrations above the limit of detection for at least 20% of measurements. Boxplots are separated by cases (dark grey), housemate controls (light grey) and matched controls (grey). The x-axis provides the compounds and the y-axis labels are displayed on the original scale even though the data were log₁₀ transformed. FIG. 10 illustrates graphical views of boxplots of log₁₀ (concentrations) for quantifiable VOCs with concentrations above the limit of detection for at least 20% of measurements. Boxplots are separated by cases (dark grey) and housemate and matched controls combined (light grey). The x-axis provides the compounds and the y-axis labels are displayed on the original scale even though the data were log₁₀ transformed.

TABLE 15
Prediction performance of log concentration of quantifiable
VOCs that have at least 20 percent concentration measurements
above the limit of detection. Performance is assessed as AUC
in single-variable models and is reported in the training and
test data. Ranking based on training data. The column labeled
N indicates the number of samples used in the model

| | |
|---|---|
| Train | Test |

| | Individual | | Individual | |
|---|---|---|---|---|
| | AUC | N | AUC | N |
| Acetoin | 0.649 | 77 | 0.650 | 141 |
| Heptanal | 0.610 | 68 | 0.511 | 138 |
| Dodecane | 0.574 | 80 | 0.541 | 141 |
| Cyclohexanone | 0.509 | 60 | 0.515 | 17 |
| 2-Pentanone | 0.502 | 76 | 0.590 | 132 |

Table 15 provides individual VOCs S1LC case prediction performance using univariate logistic regression based on log concentrations above the limit of detection. Acetoin, Heptanal have training AUC greater than 0.6, while other compounds have AUCs close to 0.5. The AUC for Acetoin is 0.649 in the training data and 0.650 in the testing data. In contrast, the AUC for Heptanal is 0.610 in the training data, but falls to 0.511 in the test data. Dodecane has a consistent AUC across training and test data (0.574 in training and 0.541 in testing).

A forward selection approach was used to identify the combination of most predictive VOCs. Selection of VOCs and ranking of models were based on the maximum improvement in the AUC using training data. For each selected model the AUC on the test data was also computed. Missing observations are excluded when individual VOCs are below the detection limit in each candidate model. Table 16 displays the results of the procedure and provides both the training and test AUC as additional covariates are included into the model. The table is cumulative; for example, the row labeled 2-Pentanone indicates that 2-Pentanone was the third variable added to the model and the corresponding AUC refers to the model that includes Acetoin, Heptanal, and 2-Pentanone.

In the log concentration analysis, Acetoin is the strongest predictor with a training AUC of 0.649 and a test AUC of 0.65. Adding Heptanal increases the training AUC to 0.669 and decreases the test AUC to 0.669. Adding 2-Pentanone to the model increases slightly the training AUC (from 0.669 to 0.689) though the test AUC of 0.601 is still below the test AUC of 0.65 for Acetoin alone. This suggests that using a one variable model based on Acetoin may be the best approach. One could also consider a two variable model adding either Dodecane or 2-Pentanone. However, more complex models are not considered at this time given the results in Table 16 and the high correlations among the other log concentrations of quantifiable VOCs shown in Table 14.

TABLE 16
Forward selection results based on quantifiable VOC log concentrations.
Each row indicates a cumulative model; for example, the row labeled Dodecane
correspond to a model that includes Acetoin, Heptanal and Dodecane. Ranking
is based on training AUC (both training and test AUCs are shown). The column
labeled N indicates the number of samples used in the model

| | Univariate model | | | | Forward selection cumulative model | | | |
|---|---|---|---|---|---|---|---|---|
| | Training | | Test | | Training | | Test | |
| | Univariate | | Univariate | | Cumulative | | Cumulative | |
| VOC | AUC | N | AUC | N | AUC | N | AUC | N |
| Acetoin | 0.649 | 77 | 0.650 | 141 | 0.649 | 77 | 0.650 | 141 |
| Heptanal | 0.610 | 68 | 0.511 | 138 | 0.669 | 64 | 0.559 | 137 |
| 2-Pentanone | 0.502 | 76 | 0.590 | 132 | 0.689 | 63 | 0.601 | 128 |
| Dodecane | 0.574 | 80 | 0.541 | 141 | 0.686 | 63 | 0.592 | 127 |

TABLE 17
Results for t-tests comparing the mean of the log concentration between cases and
combined controls. Results are shown for the training, test, and combined data

| | | | | | | | Combined data | | |
|---|---|---|---|---|---|---|---|---|---|
| | Training data | | | Test data | | | (training + test) | | |
| | N | N | p- | N | N | p- | N | N | p- |
| VOC | cases | controls | value | cases | controls | value | cases | controls | value |
| 2-Pentanone | 28 | 48 | 0.568 | 55 | 77 | 0.105 | 83 | 125 | 0.699 |
| Acetoin | 28 | 49 | 0.091 | 57 | 84 | 0.001 | 85 | 133 | <0.001 |
| Dodecane | 29 | 51 | 0.268 | 56 | 85 | 0.462 | 85 | 136 | 0.762 |
| Heptanal | 23 | 45 | 0.084 | 56 | 82 | 0.837 | 79 | 127 | 0.237 |

Un-paired t-tests were conducted to compare the mean of the log concentration among cases and combined controls separately in the training and test data as well as in the combined test and training data. Table 17 provides the results indicating that the difference in log concentrations of Acetoin is: (1) not significant at the α=0.05 level in the training sample (p-value=0.091; (2) is significant in the test sample (p-value=0.001); and (3) is significant in the combined sample (p-value=<0.001). This is likely due to the differences in sample sizes between the training and testing data sets. For all other VOCs and data sets, the differences were not statistically significant at the α=0.05 level.

Results based on VOC concentrations suggest that Acetoin: (1) has most concentrations above the limit of detection; (2) leads to the best predictive model in the test data; and (3) has a stable performance when transitioning from training to test data. Thus, the specific Acetoin concentration thresholds expressed in μg/L and their associated S1LC case prediction performance are explored. Because Acetoin concentrations were, on average, lower in S1LC patients compared to controls, the test follows the following rule:

$$\{ \begin{matrix}if {Acetoin}^{test} < {10}^{threshold} train , participant is classified as case ; \\ if {Acetoin}^{test} \geq {10}^{threshold} train , participant is classified as control .\end{matrix}$$

The thresholds, thresholdₜᵣₐᵢₙ, can be chosen in many different ways to balance sensitivity and specificity. Here, the following thresholds on the percentiles of Acetoin concentrations in the training data of controls are considered: (a) the 10th percentile (0.026 μg/L); (b) the 25th percentile (0.044 μg/L); and the 50th percentile (0.098 μg/L). These thresholds are provided directly on the concentration scale. The corresponding thresholds, thresholdₜᵣₐᵢₙ, on the log₁₀ concentration scale can be obtained by taking the log₁₀ transformation of the thresholds on the concentration scale. These choices are made for illustration purposes only.

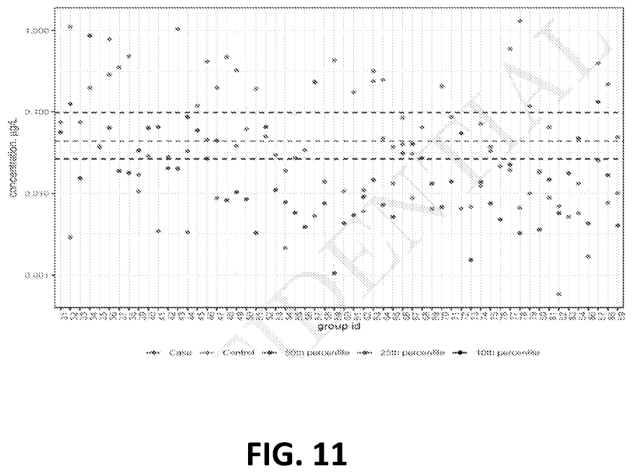

FIG. 11 displays the Acetoin concentration for each biopsy-confirmed S1LC case (dark grey dot) and control (grey dot). The x-axis is the test group number starting from 31 because the first 30 groups were used for training. On each vertical line there are either: (1) two dots (one dark grey and one grey), when the group contains a biopsy-confirmed S1LC case and a matched control; or (2) three dots (one dark grey and two grey) when the group contains a biopsy confirmed S1LC case, a matched control, and a housemate control. For example, group 31 has two dots and group 32 has three dots (dots shown on vertical lines). The y-axis is labeled on the scale of the concentration (μg/L), even though data was log₁₀ transformed for visualization purposes. The dashed horizontal lines correspond to the classification thresholds based on the distribution of Acetoin concentration in controls in the training data set: 10^{th} percentile shown in black (0.026 μg/L), 25th percentile shown in light grey (0.044 μg/L) and 50^{th} percentile (0.098 μg/L) shown in magenta. For each threshold, study participants below the corresponding line are classified as cases and above the line as controls. In summary, the color of the dots is the true S1LC case status (dark grey cancer, grey), while the position of the dot relative to one of the horizontal lines is the prediction of S1LC case status (below cancer, above control). This Figure provides the visual tradeoff in terms of false positives and false negative predictions as a function of the threshold on Acetoin concentrations.

Table 18 further quantifies the results displayed in FIG. 11. The part of the table labeled “Test Data” corresponds exactly to FIG. 11 (test data), while the part labeled “All Data” corresponds to the combination of training and test data (corresponding figure not shown). For example, consider the scenario when S1LC cases are predicted when Acetoin concentration is below 0.026 μg/L. When the test data are used, 37 S1LC cases and 49 controls are correctly identified, 20 cases are incorrectly classified as controls and 35 controls are incorrectly classified as cases. When all data are used (cases and controls) 44 S1LC cases and 93 controls are correctly identified, 41 cases are incorrectly classified as controls and 40 controls are incorrectly classified as cases.

FIG. 11 illustrates a graphical view of classification based on Acetoin concentration threshold using the test data. The x-axis is the group number (starting at 31 because the first 30 groups are for training), each group with either two or three study participants. The y-axis is labeled on the concentration scale (μg/L), but data are log₁₀ transformed. Each point is a study participant (dark grey S1LC case, grey control). Horizontal lines correspond to three thresholds based on percentiles of the Acetoin concentrations distribution in all training data controls: 10th (0.026 μg/L, shown in black), 25th (0.044 μg/L, shown in light grey) and 50th (0.098 μg/L, shown in magenta). For each threshold, participants below the line are classified as cases and above the line as controls.

TABLE 18
Classification table for three Acetoin concentration thresholds
using the Test data and All data (training + test)

| | | | Test data | | All data | |
|---|---|---|---|---|---|---|
| | threshold | type | Case | Control | Case | Control |
| predicted | 10% (0.026 μg/L) | Case | 37 | 35 | 44 | 40 |
| | | Control | 20 | 49 | 41 | 93 |
| | 25% (0.044 μg/L) | Case | 43 | 48 | 57 | 61 |
| | | Control | 14 | 36 | 28 | 72 |
| | Median (0.098 μg/L) | Case | 53 | 60 | 74 | 84 |
| | | Control | 4 | 24 | 11 | 49 |

TABLE 19
Estimated sensitivity (proportion of correctly identified SILC cases), specificity
(proportion of correctly identified controls), and accuracy (proportion
of correctly classified cases and controls) for three Acetoin concentration
thresholds using the Test data and All data (training + test)

| | Test data | | | All data | | |
|---|---|---|---|---|---|---|
| Threshold | Sensitivity | Specificity | Accuracy | Sensitivity | Specificity | Accuracy |
| 10% (0.026 μg/L) | 0.649 | 0.583 | 0.610 | 0.518 | 0.699 | 0.628 |
| 25% (0.044 μg/L) | 0.754 | 0.429 | 0.560 | 0.671 | 0.541 | 0.592 |
| 50% (0.098 μg/L) | 0.930 | 0.286 | 0.546 | 0.871 | 0.368 | 0.564 |

Table 19 provides the estimated sensitivity (proportion of correctly identified S1LC cases), specificity (proportion of correctly identified controls), and accuracy (proportion of correctly classified cases and controls). The part of the table labeled “Test Data” corresponds exactly to FIG. 11 (test data), while the part labeled “All Data” corresponds to the combination of training and test data (corresponding figure not shown). There is a direct correspondence between Tables 19 and 18. For example, consider the scenario when S1LC cases are predicted when Acetoin concentration is below 0.026 μg/L. When the test data are used sensitivity was 0.649=37/(37+20), specificity was 0.583=49/(49+35) and accuracy was 0.61=(37+49)/(37+20+49+35).

Focus has been on the prediction performance of concentrations when they are above the limit of detection, which was the main goal of the study. However, several VOCs have large proportions of observations that are below the limit of detection. Thus, there is a need to investigate whether being above/below the limit of detection predicts S1LC status. To conduct this analysis missing VOC concentrations were recoded as 0 and those present were recoded as 1. These recoded variables are referred to as presence/absence of individual VOCs.

TABLE 20
Missing concentrations individual VOC discriminative ability.
Prediction performance measured as area under the curve (AUC)
in the training and test data when predicting S1LC cases based
on individual binary predictors defined as “above or below
LOD” for each VOC. VOCs are ordered by name, not by any measure.

Univariate AUC

| VOC | Training | Test |
|---|---|---|
| 3-3-dimethyl-pentane | 0.504 | 0.494 |
| 2-Butanone | 0.504 | 0.503 |
| 2-Pentanone | 0.504 | 0.474 |
| Toluene | 0.524 | 0.500 |
| 3-Methyl-1-Butanol | 0.561 | 0.518 |
| Acetoin | 0.514 | 0.497 |
| 2-Hexanol | 0.555 | 0.486 |
| Hexanal | 0.528 | 0.500 |
| Ethylbenzene | 0.504 | 0.511 |
| Heptanal | 0.558 | 0.494 |
| Cyclohexanone | 0.547 | 0.488 |
| p-Cymene | 0.630 | 0.580 |
| Dodecane | 0.517 | 0.511 |

Analyses were conducted using individual quantifiable VOCs presence/absence data as predictors and S1LC case indicators as outcome. Table 20 provides the train and test data AUC for each VOC presence/absence data. All models are univariate (using one presence/absence predictor). The test AUCs for all compounds, except p-Cymene are close to 0.5. The AUC for p-Cymene is 0.633 in the training data and 0.580 in the test data. The limit of detection for p-Cymene (see Table 21) was 0.00011 μg/L. The model uses a decision rule of having a p-Cymene breath concentration below 0.00011 μg/L to predict S1LC cases.

Analysis of VOC concentrations data indicated that Acetoin was the strongest predictor S1LC cases in the test data set. Analysis of presence/absence concentrations data indicated that p-Cymene being below the limit of detection was predictive of S1LC. Here, the investigation is focused on whether the combination of Acetoin and presence/absence of p-Cymene given its specific LOD in or study performs better than Acetoin alone.

Results indicate that the model with Acetoin alone has better prediction performance (training AUC=0.649; testing AUC=0.65) than the model with Acetoin and the indicator variable for presence/absence of p-Cymene (training AUC=0.606; testing AUC=0.504).

TABLE 21
Compounds concentration range, limit of detection,
and upper bound of calibration curve. This is for
all data (train and test). All values are in μg/L

| | | | limit of | upper |
|---|---|---|---|---|
| compound | min | max | detection | bound |
| 2-Butanone | 0.00922 | 0.13602 | 0.00815 | 0.40000 |
| 2-Hexanol | 0.00219 | 0.08200 | 0.00199 | 0.00312 |
| 2-Pentanone | 0.00133 | 0.22125 | 0.00130 | 0.10000 |
| 3-Methyl-1-Butanol | 0.00184 | 0.04532 | 0.00181 | 0.05000 |
| 3,3-dimethyl pentane | 0.00157 | 0.00827 | 0.00124 | 0.10000 |
| Acetoin | 0.00059 | 17.49725 | 0.00037 | 4.00000 |
| Cyclohexanone | 0.00593 | 0.21534 | 0.00581 | 0.10000 |
| Dodecane | 0.00031 | 0.08950 | 0.00002 | 0.02500 |
| Ethylbenzene | 0.00082 | 0.00158 | 0.00074 | 0.05000 |
| Heptanal | 0.00028 | 0.13747 | 0.00023 | 0.02500 |
| Hexanal | 0.00335 | 0.02539 | 0.00334 | 0.10000 |
| p-Cymene | 0.00011 | 0.01693 | 0.00011 | 0.05000 |
| Toluene | 0.01864 | 0.03048 | 0.01854 | 0.20000 |

Table 21 provides the range of the distribution of detected concentrations for Bag 2 in all analyzed data (testing and training combined) and the corresponding limit of detection for every compound. All values are expressed in μg/L. For example, for 2-Pentanone the minimum observed concentration was 0.00133 μg/L and the maximum observed concentration was 0.22125 μg/L with a limit of detection of 0.00130 μg/L and an upper bound for the concentration curve calibration of 0.10000 μg/L. It is worth noting that most limits of detection are in the nanograms (one thousandth of one microgram) per liter (ng/L) range. The highest limit of detection among the thirteen quantifiable compounds in this study is Toluene, with a limit of detection of 0.01854 or approximately, 18 ng/L.

The maximum upper bound for concentration for each compound is related to the data available for calibrating the curves. A few observations were estimated to be above the upper bound and were based on extrapolation of the calibration curve. All analyses were based on data using these few extrapolated values. Two sensitivity analyses were conducted by: (1) removing all observations that were above the upper bound of concentrations; and (2) removing all observations that were more than 20% above the upper bound. Results were robust to these changes in the data, most likely because very few data points were affected by this problem.

Focus has been on thirteen quantifiable VOCs, which were identified from literature as potential predictors of cancer and for which calibration (transformation from peak area to concentrations) was possible. These are referred to as quantifiable compounds, though the term is specific to the analysis and report as the number and type of VOCs that are quantifiable can vary with the study. However, there is a large number of VOCs that were not calibrated in the data. More precisely, they have an associated peak area measurement, but do not a corresponding concentration expressed in international units of measurement. These VOCs will be referred to as “unquantifiable” VOCs, though, the list of VOCs that are not quantifiable can vary substantially from study to study.

In the study, Tentatively Identified Compounds (TICs) information was used for the unquantifiable VOC analysis. This information was obtained directly from a Chromeleon CDS system (Version 7.2.8 with NIST MS search V.2.0, Thermo Fisher Scientific). As mentioned in the EPA TIC (2006) document: “The [TICS] identification is not considered “absolute” or “confirmed” until a known standard for the suspect compound can be analyzed on the same instrument which made the tentative identification.” Due to various constraints this was not done for the unquantifiable VOCs in the study, though it was done for the 13 quantifiable VOCs.

Before the study started it was not known what and how many additional VOCs will be identified and in what proportion of the study participants each VOC will be present. Here an exploratory analysis of the unquantifiable VOCs identified in the study is provided. The statistical analysis mirrors the one conducted for the log peak area of quantifiable compounds and quantifies: (1) the association between presence/absence of each VOC and the S1LC case indicator; and (2) the association between log₁₀ peak corresponding to each VOC and the S1LC case indicator. The same training/test data split used for the analysis of quantifiable VOCs was used for the unquantifiable VOCs. For prediction purposes only data based on Bag 2 (alveolar) was used, though some summary statistics are presented for Bag 1 (tidal), as well.

In the case of quantifiable compounds only one peak area was returned by the software and calibrated to concentrations. However, for some unquantifiable VOCs sometimes there are multiple peak areas that are associated with the same compound. In these cases the area of the maximum peak was used and the other peaks were discarded. Additional analysis could be conducted using the sum of the areas or a repeated measures analysis. Only VOC peaks that were identified as being “excellent” were retained based on the criterion that both the Similarity Index (SI) and the Reverse Search Index (RSI) are greater than or equal to 900.

TABLE 22
Number of compounds that are present in each bag and the
total number of distinct compounds across two bags, by
compound quality. Training and test data combined.

| Compound | | | |
|---|---|---|---|
| quality | N Bag 1 | N Bag 2 | N distinct |
| any | 129 | 144 | 167 |
| excellent | 23 | 22 | 24 |
| good | 48 | 55 | 60 |
| insufficient | 128 | 143 | 166 |

There were 167 total VOCs with identified peaks, out of which 60 were identified as “good” (both SI and RSI greater than or equal to 800), and 24 compounds with “excellent” data (both SI and RSI greater than or equal to 900). These numbers contain VOCs in either bag that were identified in at least one study participant in all included data (training and test combined). Results are summarized in Table 22.

However, the number of VOCs identified in the breath of at least one individual was different depending on the bag. For example, there were 129 total VOCs in Bag 1 compared to 144 in Bag 2, 48 VOCs of “good” quality in Bag 1 compared to 55 in Bag 2, and 23 VOCs of “excellent” quality in Bag 1 compared to 22 in Bag 2.

Recall that the training data consists of 30 groups with a total of 81 study participants, with 30 cases and 51 combined matched and housemate controls. The test data consists of 58 groups with a total of 144 study participants, with 58 cases and 86 combined matched and housemate controls.

Table 23 provides the results for Fisher's exact test of the null hypothesis of no association between the presence/absence indicator of individual VOCs and S1LC case status in the training data for Bag 2. The column labeled N present denotes the number of cases and controls that have the specific VOC present among the 81 study participants (ncases=30; ncontrols=51) Bag 2 training data. Results are shown for VOCs with a p-value for Fisher's exact test less than 0.5 (not 0.05) for exploratory reasons. VOCs are ranked from the smallest (stronger evidence against the null hypothesis) to the largest p-value. The columns labeled “Sensitivity” and “Specificity” provide the sensitivity and specificity of the test that predicts a S1LC case if the VOC is present in the training data.

TABLE 23
Fisher's exact test of the null hypothesis of no association between the presence/absence
indicator of individual VOCs and S1LC case status. N present: number of participants
that have the VOC present among the 30 cases and 51 controls. Results are shown
for VOCs with a p-value for Fisher's exact test less than 0.5, with corresponding
sensitivity and specificity. An individual is predicted to be a S1LC case if the
VOC is present. All results are based on the training data for Bag 2.

| Compound | N present | P-value | Sensitivity | Specificity |
|---|---|---|---|---|
| Argon | 62 | 0.032 | 0.90 | 0.31 |
| Cyclopropane, ethylidene- | 25 | 0.082 | 0.43 | 0.76 |
| Isopropyl Alcohol | 21 | 0.117 | 0.37 | 0.80 |
| Cyclohexanol, 1-methyl-4-(1-methylethyl)- | 4 | 0.291 | 0.00 | 0.92 |
| Acetone | 27 | 0.342 | 0.40 | 0.71 |
| Carbon dioxide | 36 | 0.356 | 0.37 | 0.51 |
| Ethanol | 18 | 0.418 | 0.17 | 0.75 |
| Phosphonic acid, (p-hydoxyphenyl)- | 20 | 0.432 | 0.30 | 0.78 |

Table 24 provides the AUC for the training and test data for the presence/absence data for the top eight unquantifiable compounds in the study. Ranking was based on the p-values of the Fisher's exact test for no association between presence/absence and S1LC case status in the training data. Current software implementations of AUC (as implemented in the function prediction in R package ROCR) are used, though this may be inappropriate for binary predictors. A better measure of AUC is estimating the AUC without adding in ties, which tends to provide lower values of AUC. However, this version of AUC is used to keep the AUC calculations consistent within this report.

With the exception is Argon (Training AUC=0.607, Test AUC=0.509), there is good agreement between the training and test AUC. This may be due to the fact that for binary prediction there is no tuning parameter (decision threshold). Thus, the consistency of AUCs is a consequence of the stability of missing VOC proportions in the training and test data. The presence/absence of the VOCs listed in Table 24 could be potentially useful for building prediction models for S1LC cancer cases. However, the definition of presence/absence depends substantially on the technology used and its VOC detection sensitivity. In the absence of information about limits of detection and calibration curves this information cannot be directly generalizable.

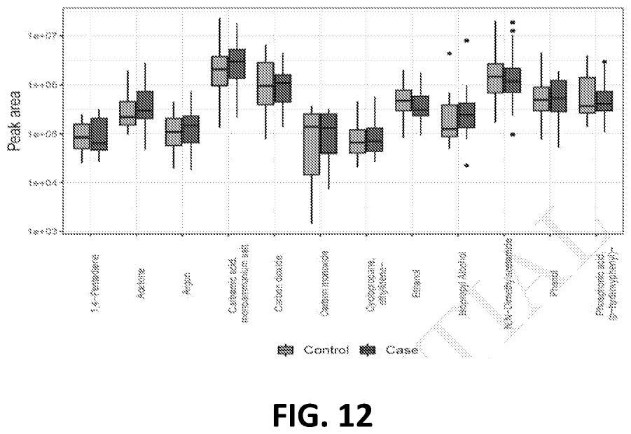

In this section the prediction performance of the log₁₀ peak area of unquantifiable VOCs for S1LC case status is explored. Only VOC peaks that were identified as being of “excellent” quality (SI and RSI greater than or equal to 900) are used. FIG. 12 displays the boxplots of the log₁₀ peak areas in the training data for VOCs that had at least 5 cases and 5 controls with data of “excellent” quality. The grey boxplots correspond to combined matched and housemate controls and dark grey boxplots correspond to biopsy-confirmed S1LC cases. In the training data set some of the unquantifiable VOCs have higher log₁₀ peak areas in S1LC cases than controls; see, for example, Acetone, Argon, Carbamic Acid, Carbon Dioxide, and Isopropyl Alcohol. However, other unquantifiable VOCs have lower log₁₀ peak areas in S1LC cases than controls; see, for example, 1,4-Pentadiene, Ethanol, and N,N-Dimethylacetamide.

TABLE 24
P-values and AUCs for presence/absence predictors
of S1LC cases in the training and testing data.

| | p-value | | AUC | |
|---|---|---|---|---|
| Compound | Test | Training | Test | Training |
| Argon | 0.782 | 0.032 | 0.509 | 0.607 |
| Cyclopropane, ethylidene- | 0.313 | 0.082 | 0.544 | 0.599 |
| Isopropyl Alcohol | 0.025 | 0.117 | 0.596 | 0.585 |
| Carbon dioxide | 0.237 | 0.356 | 0.552 | 0.562 |
| Acetone | 0.171 | 0.342 | 0.563 | 0.553 |
| Ethanol | 0.310 | 0.418 | 0.546 | 0.544 |
| Phosphonic acid, | 0.125 | 0.432 | 0.570 | 0.542 |
| (p-hydroxyphenyl)- | | | | |
| Cyclohexanol, 1- | 0.273 | 0.291 | 0.517 | 0.539 |
| methyl-4(1-methylethyl)- | | | | |

Table 25 displays the S1LC case prediction performance of login peak area of unquantifiable VOCs based on t-tests and AUCs. VOCs are ranked from the smallest to the largest p-value for the t-test and only VOCs with an AUC larger than 0.55 are shown. Also shown are the number of samples available for each compound broken down by case status. Table 26 displays similar results with Table 25, but includes VOCs that had an AUC greater than 0.55 in either the training or test data sets. VOCs are ranked from the largest to the smallest AUC in the test data.

Phosponic acid has a large training AUC (0.838), but this is based on a small number of study participants who had this particular VOC detected (9 cases and 11 controls). In the test data the AUC for Phosphonic acid is much smaller (0.538) based on a larger number of study participants who had this particular VOC detected (31 cases and 34 controls). Carbamic acid (training AUC=0.637, test AUC=0.595), Acetone (training AUC=0.572, test AUC=0.698), Carbon dioxide (training AUC=0.658, test AUC=0.512), and Cyclopropane (training AUC=0.571, test AUC=0.532) have been identified as possible targets for further investigation. All compounds in Table 26 could be of interest in future analyses.

Overall, a list of promising unquantifiable VOC based both on the presence/absence and on the compound and peak area are identified. To evaluate the translational potential of these findings additional studies would need to be conducted, including developing calibration curves to transform peak area values into concentrations and independent validation studies. Given the experience with quantifiable VOCs, results may or may not be reproducible depending on the limits of detection and the patterns of missingness induced by technological limitations.

FIG. 12 illustrates graphical views of boxplots of log₁₀(peak) for unquantifiable VOCs separated by cases (dark grey), housemate and matched controls combined (grey). The x-axis are the compounds and the y-axis labels are displayed on the original scale even though the data were log₁₀ transformed.

TABLE 25
Training data: area under the curve (AUC) and p-values for unpaired t-tests
for prediction of S1LC case status from individual unquantifiable VOC logn₁₀ peak
areas. Mean: mean log₁₀ peak areas in cases and controls, respectively. Number
number of study participants with a particular VOC among case, controls, and
combined. VOCs are ranked participants with a particular VOC among cases,
controls, and combined. VOCs are ranked from the largest to smallest ACU in
the training data and only VOCs awith AUC larger than 0.55 are shown. VOCs
are ranked from the largest to smallest AUC in the training data

| | | | Mean | | Number | | |
|---|---|---|---|---|---|---|---|
| Compound | AUC | p-value | Case | Control | Case | Control | Total |
| Phosphonic acid, | 0.838 | 0.023 | 5.87 | 6.16 | 9 | 11 | 20 |
| (p-hydroxyphenyl)- | | | | | | | |
| Carbon dioxide | 0.658 | 0.154 | 5.94 | 6.20 | 11 | 25 | 36 |
| Car [symbol] acid, | 0.637 | 0.0 [symbol] 8 | 6.77 | 6. [symbol] 5 | 26 | 44 | 70 |
| monoammonium salt | | | | | | | |
| Acetone | 0.572 | 0.808 | 5.75 | 5.71 | 12 | 15 | 27 |
| Cyclopropane, ethyl [symbol] | 0.571 | 0.564 | 5.31 | 5.25 | 13 | 12 | 25 |

[symbol]

indicates data missing or illegible when filed

TABLE 26
Test data: area under the curve (AUC) and p-values for unpaired t-tests for prediction
of SILC case status from individual unquantifiable VOC log₁₀ peak areas. Mean: mean
log₁₀ peak areas in cases and controls, respectively. Number: number of study
participants with a particular VOC among cases, controls, and combined. VOCs
are ranked from the largest to smallest AUC in the training data. Only VOCs
with AUC larger than 0.55 in either the training or test data are shown. VOCs
are ranked from the largest to smallest AUC in the test data

| | | | Mean | | Number | | |
|---|---|---|---|---|---|---|---|
| Compound | AUC | p-value | Case | Control | Case | Control | Total |
| Isopropyl Alcolhol | 0.78 [symbol] | <0.001 | 5.3 [symbol] | [symbol] .08 | 30 | 28 | 58 |
| [symbol] , | 0.750 | 0.443 | 4.87 | 4.69 | 4 | 2 | 6 |
| 1,1,1,5,5,hexamethyl | | | | | | | |
| Acetone | 0. [symbol] 98 | 0.00 [symbol] | 5. [symbol] 0 | [symbol] .33 | 37 | 44 | 81 |
| Ethanol | 0.604 | 0.22 [symbol] | 5.46 | 5.55 | 31 | 38 | 69 |
| Carb [symbol] acid, | 0. [symbol] | 0.08 [symbol] | 0.24 | 6.12 | 44 | 68 | 112 |
| monoammonium salt | | | | | | | |
| Phosphonic acid, | 0. [symbol] | 0. [symbol] 2 | [symbol] .00 | [symbol] .60 | 31 | 34 | 65 |
| (p-hydroxyphenyl) [symbol] | | | | | | | |
| Cyclopropane, | 0. [symbol] 3 [symbol] | 0. [symbol] | 4.74 | 4.77 | 30 | 37 | 67 |
| ethyl [symbol] | | | | | | | |
| Carbon dioxide | 0. [symbol] 12 | 0.947 | [symbol] 90 | 5. [symbol] | 27 | 49 | 76 |

[symbol]

indicates data missing or illegible when filed

Many VOCs in exhaled breath had low concentrations in the range of 0.0001 to 17.4973 μg/L for Acetoin and 0.00011 to 0.22125 μg/L for all other VOCs. Each VOC had a different LOD and the percent of VOC measurements below the LOD for most VOCs was high for combined, training, and test data. Among the thirteen quantifiable VOCs considered in this analysis, only four VOC were below the LOD in less than 10% across all samples: 2-Pentanone (7.6%), Acetoin (3.1%), Heptanal (8.4%), Dodecane (1.8%). The proportion of VOCs below LOD among cases and controls in testing and training data was similar for all VOCs except p-Cymene. For p-Cymene the percentage of compounds below LOD was higher in S1LC cases. In the training data, 64% of the measurements were below LOD among cases and 38% among controls. In the test data 70% of the measurements were below LOD among cases and 54% among controls.

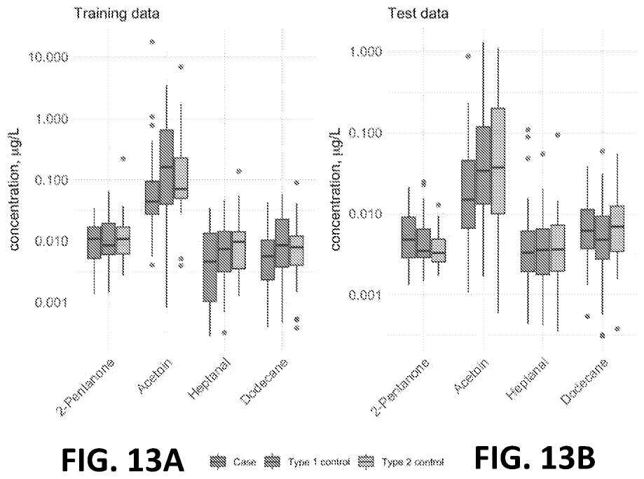

FIGS. 13A and 13B illustrate graphical views of the distributions of VOC concentrations for training (FIG. 13A) and test (FIG. 13B) data separated by cases and control types. Acetoin concentrations tended to be lower both in training and test cases, while Heptanal and Dodecane concentrations tended to be lower in training and roughly similar in test samples. 2-Pentanone concentrations tended to be higher in both training and test cases than controls, though the difference was not significant (combined data t-test p-value=0.699; see Table 17).

As several VOCs had large proportions of observations that are below the LOD, the predictive performance was investigated for every VOC being above/below the limit of detection (LOD). Univariate analyses of the prediction performance of S1LC cases using the predictors “above or below the LOD” indicated that p-Cymene had the highest predictive accuracy (training AUC=0.630; testing AUC=0.580; see Table 20). The limit of detection for p-Cymene was 0.00011 μg/L; thus, the model uses a decision rule of having a p-Cymene breath concentration below 0.00011 μg/L to predict S1LC cases. The test AUCs for the remaining 12 VOCs was close to 0.5 indicating that being above or below the LOD was not predictive of S1LC.

Table 17 presents the results of comparing the mean of the log₁₀ concentration among cases and combined controls in the training, test, and combined test and training data using unpaired t-test. With the exception of Acetoin, the difference between cases and controls was not statistically significant for any of the group comparisons. For Acetoin the difference in the means was: (1) not significant in the training sample (p-value=0.091); (2) significant in the test sample (p-value=0.001); and (3) significant in the combined sample (p-value<0.001). These differences are likely due to the difference in sample size; for example, for Acetoin there are 28 cases and 49 controls in the training data, but there are 85 cases and 133 controls in the combined data.

Table 16 provides individual VOCs S1LC case prediction performance using univariate and multivariate forward selection logistic regression based on log₁₀ concentrations above the LOD. In univariate models (one predictor at a time) Acetoin and Heptanal have training AUC greater than 0.6, while other compounds have AUCs close to 0.5. The AUC for Acetoin is 0.649 in the training data (N=77) and 0.650 in the test data (N=141), indicating that the predictive performance of Acetoin was preserved in the test data. In contrast, the AUC for Heptanal is 0.610 in the training data (N=68) and only 0.511 in the test data (N=138), indicating that Heptanal may not be a reliable predictor of S1LC cases. Dodecane has a consistent, low AUC for training (0.574) and test (0.541) data.

Cumulative AUCs for the multivariate forward selection logistic regression as additional VOCs are included into the model are provided in Table 16 for both the training and test data. Acetoin is the strongest predictor with a training AUC of 0.649 and a test AUC of 0.650. Adding Heptanal increases the training AUC to 0.669 and decreases the test AUC to 0.559. Adding 2-Pentanone to the model increases the training AUC (from 0.669 to 0.689) though the test AUC of 0.601 is lower than the test AUC of 0.65 for Acetoin alone. A two variable model adding either Dodecane or 2-Pentanone could also be considered. However, more complex models are not considered at this time given the low individual AUC values for these VOCs and the high correlations among the other log concentrations of VOC (Table S3 in the supplementary materials).

Results based on VOC concentrations suggest that Acetoin: (1) has most concentrations above the limit of detection; (2) leads to the best predictive model in the test data; and (3) has a stable performance when transitioning from training to test data. Thus, the specific Acetoin concentration thresholds expressed in mg/L and their associated S1LC case prediction performance are examined. Because Acetoin concentrations were, on average, lower in S1LC patients compared to controls, the test follows the following rule:
if Acetoinₜₑₛₜ<10^{threshold(from training data)} participant is classified as S1LC case;
if Acetoinₜₑₛₜ³ 10^{threshold(from training data)} participant is classified as control.

The threshold (from training data), can be chosen in many different ways to balance sensitivity and specificity. Here the following thresholds were considered based on the percentiles of Acetoin concentrations in the training data of controls: (a) the 10th percentile (0.026 mg/L); (b) the 25th percentile (0.044 mg/L); and the 50^{th} percentile (0.098 mg/L).

This was the largest case-control VOC study to date with the inclusion of a healthy control and a housemate control to aid in the elimination of potential environmental confounders for VOCs that may indicate the presence of lung cancer. The control group (S1LC) cases was diverse in terms of covariates and analytic approach of combining type 1 and 2 cases ensures that study results are generalizable to the population. The novelty of the study consists of its focus on: (1) early lung cancer detection, specifically S1LC; (2) practical, translatable and reproducible signature of breath VOC for S1LC; (3) design of experiment targeted to elimination of potential confounders due to environment, technology, and breath analysis procedure; and (4) definition of training and testing data sets before data were collected. The data presents results that are contrary to the published literature indicating that: (a) most VOCs published in the literature have a weak or inexistent association with S1LC; (b) Acetoin, the only VOC that was associated with S1LC, has a much lower predictive performance than the performance of previously published VOC signatures, though none of these results specifically focused on S1LC; and (c) Acetoin concentrations were on average lower (not higher) in the breath of S1LC cases than in controls. Acetoin has an AUC of 0.65 with a sensitivity of 87.1% (specificity of 36.8%) when predicting that a person has SILO if the Acetoin concentration is below 0.098 mg/L. This is a promising result that will need further investigation as this single VOC approaches the sensitivity of LDCT⁴.

Acetoin has not been a VOC closely studied in its relationship to lung cancer, and in a recent review article on VOCs it was not a described candidate VOC for the detection of lung cancer but is typically used in the flavorings of foods, as well as e-cigarettes. As additional VOCs were added to the model, the test AUC dropped. This is in contrast to multiple other studies. Indeed, in a small study of seventy patients, a signature was identified without providing the specific VOCs, with a sensitivity of 81% and specificity of 91%. A prior study of 229 participants reported an AUC of 0.81, though the VOCs used were not disclosed. Another studied 2-butanone, 3-hydroxy-2-butanone, 2-hydroxyacetaldehyde and 4-hydroxyhexanal in a large study with 405 participants, and were able to show a sensitivity and specificity of 93.6% and 85.6%, respectively. There are concerns about these studies, especially because: (1) the data are not available; (2) methods used are only superficially described; (3) analytic methods used can be over-fit; (4) VOC measurements are not expressed in concentration units, which implies that the measurement values may be indistinguishable from the experimental noise; and (5) there are many levels of data processing and cleaning that cannot be understood when data and code are not reproducible.

There are several limitations to this trial. First, the presence of dead space in the lung can dilute VOC's in the same breath. To combat this, a separate Tedlar® bag was used for the first 150-200 cc of exhalation, followed by the rest of the breath into a 1 L Tedlar® bag. Second, the effect of condensation on VOCs is unknown and, unfortunately, this effect was not controllable in the Tedlar® bags. Third, it is not possible to control for all environmental exposures, so there may be confounders present that were not considered—this includes the potential that participants did not abstain from smoking, vaping or drinking prior to breath collection. Fourth, although the protocol planned to analyze all breaths within a 24-hour period, this was not always the case. It is possible that these delays could have led to changes in the VOC concentrations in the Tedlar® bags. Fifth, S1LC was the focus, which may not be associated with substantial changes in breath VOCs. This leaves the possibility that changes may occur in more advanced stages of lung cancer. Sixth, the time interval to abstain from smoking, vaping, or drinking for at least 30 minutes prior to collecting exhaled breath interval was chosen as a reasonable compromise for the participants and the study feasibility, however different interval lengths could affect the concentration of individual VOCs. Last, many of the demographic confounders were based on recall, such as a family history of cancer-selective memory may have played a part in answers when participants are being biopsied to assess whether they have cancer or not.

Lung cancer is the number one cause of cancer related deaths in the United States¹. The 5-year survival of patients identified to have lung cancer drastically decreases with each advancing stage. In the most recent American Cancer Society statistics, the 5-year survival for localized, regional and distant was 61%, 35%, 6%, respectively. Given the drastic decrease in survival for every increasing stage, a minimally invasive, accurate diagnostic test is needed.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A method of detecting stage one lung cancer in a subject comprising:
receiving a breath sample from the subject;
analyzing the breath sample to detect at least one of Acetoin, Dodecane, and p-Cymene;
initiating a follow-up plan for the subject, if the at least one of Acetoin, Dodecane, and p-Cymene is detected.

2. The method of claim 1 further comprising receiving multiple breath samples from the subject.

3. The method of claim 1 further comprising using a device for analysis of the VOCs in the breath.

4. The method of claim 3 further comprising using the device more than once, in order to confirm results.

5. The method of claim 1 further comprising collecting the breath sample in a bag or other receptacle.

6. The method of claim 5 wherein the bag or other receptacle comprises a Tedlar® bag or other film bag.

7. The method of claim 1 further comprising analyzing the breath sample within 24 hours of collection.

8. The method of claim 1 further comprising analyzing the breath sample within 2 hours of collection.

9. The method of claim 1 further comprising using a gas chromatograph for analysis of the breath sample.

10. The method of claim 1 wherein the follow up plan further comprises additional testing, treatment, preventative and/or lifestyle changes.

11. A method of detecting stage one lung cancer in a subject comprising:
analyzing a breath sample from the subject to detect at least one of Acetoin, Dodecane, and p-Cymene;
initiating a follow-up plan for the subject, if the at least one of Acetoin, Dodecane, and p-Cymene is detected.

12. The method of claim 11 further comprising analyzing multiple breath samples from the subject.

13. The method of claim 11 further comprising using a device for analysis of the VOCs in the breath.

14. The method of claim 13 further comprising using the device more than once, in order to confirm results.

15. The method of claim 11 further comprising analyzing the breath sample within 24 hours of collection.

16. The method of claim 11 further comprising analyzing the breath sample within 2 hours of collection.

17. The method of claim 11 further comprising using a gas chromatograph for analysis of the breath sample.
